SEQUENCE LISTING (12 sequences)

SEQ ID NO: 1 - protein, 432 aa, Homo sapiens
  misc_feature (1)..(432): glial fibrillary acidic protein
MERRRITSAARRSYVSSGEMMVGGLAPGRRLGPGTRLSLARMPPPLPTRVDFSLAGALNAGFKETRASERAEMMELNDRFASYIEKVRFLEQQNKALAAELNQLRAKEPTKLADVYQAELRELRLRLDQLTANSARLEVERDNLAQDLATVRQKLQDETNLRLEAENNLAAYRQEADEATLARLDLERKIESLEEEIRFLRKIHEEEVRELQEQLARQQVHVELDVAKPDLTAALKEIRTQYEAMASSNMHEAEEWYRSKFADLTDAAARNAELLRQAKHEANDYRRQLQSLTCDLESLRGTNESLERQMREQEERHVREAASYQEALARLEEEGQSLKDEMARHLQEYQDLLNVKLALDIEIATYRKLLEGEENRITIPVQTFSNLQIRETSLDTKSVSEGHLKRNIVVKTVEMRDGEVIKESKQEHKDVM

SEQ ID NO: 2 - protein, 758 aa, Homo sapiens
  misc_feature (1)..(758): tau isoform 1
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKHPTPGSSDPLIQPSSPAVCPEPPSSPKYVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 3 - protein, 1827 aa, Homo sapiens
  misc_feature (1)..(1827): MAP-2 isoform 1
MADERKDEAKAPHWTSAPLTEASAHSHPPEIKDQGGAGEGLVRSANGFPYREDEEGAFGEHGSQGTYSNTKENGINGELTSADRETAEEVSARIVQVVTAEAVAVLKGEQEKEAQHKDQTAALPLAAEETANLPPSPPPSPASEQTVTVEEDLLTASKMEFHDQQELTPSTAEPSDQKEKESEKQSKPGEDLKHAALVSQPETTKTYPDKKDMQGTEEEKAPLALFGHTLVASLEDMKQKTEPSLVVPGIDLPKEPPTPKEQKDWFIEMPTEAKKDEWGLVAPISPGPLTPMREKDVFDDIPKWEGKQFDSPMPSPFQGGSFTLPLDVMKNEIVTETSPFAPAFLQPDDKKSLQQTSGPATAKDSFKIEEPHEAKPDKMAEAPPSEAMTLPKDAHIPVVEEHVMGKVLEEEKEAINQETVQQRDTFTPSGQEPILTEKETELKLEEKTTISDKEAVPKESKPPKPADEEIGIIQTSTEHTFSEQKDQEPTTDMLKQDSFPVSLEQAVTDSAMTSKTLEKAMTEPSALIEKSSIQELFEMRVDDKDKIEGVGAATSAELDMPFYEDKSGMSKYFETSALKEEATKSIEPGSDYYELSDTRESVHESIDTMSPMHKNGDKEFQTGKESQPSPPAQEAGYSTLAQSYPSDLPEEPSSPQERMFTIDPKVYGEKRDLHSKNKDDLTLSRSLGLGGRSAIEQRSMSINLPMSCLDSIALGFNFGRGHDLSPLASDILTNTSGSMDEGDDYLPATTPALEKAPCFPVESKEEEQIEKVKATGEESTQAEISCESPFLAKDFYKNGTVMAPDLPEMLDLAGTRSRLASVSADAEVARRKSVPSETVVEDSRTGLPPVTDENHVIVKTDSQLEDLGYCVFNKYTVPLPSPVQDSENLSGESGTFYEGTDDKVRRDLATDLSLIEVKLAAAGRVKDEFSVDKEASAHISGDKSGLSKEFDQEKKANDRLDTVLEKSEEHADSKEHAKKTEEAGDEIETFGLGVTYEQALAKDLSIPTDASSEKAEKGLSSVPEIAEVEPSKKVEQGLDFAVQGQLDVKISDFGQMASGLNIDDRRATELKLEATQDMTPSSKAPQEADAFMGVESGHMKEGTKVSETEVKEKVAKPDLVHQEAVDKEESYESSGEHESLTMESLKADEGKKETSPESSLIQDEIAVKLSVEIPCPPAVSEADLATDERADVQMEFIQGPKEESKETPDISITPSDVAEPLHETIVSEPAEIQSEEEEIEAQGEYDKLLFRSDTLQITDLGVSGAREEFVETCPSEHKGVIESVVTIEDDFITVVQTTTDEGESGSHSVRFAALEQPEVERRPSPHDEEEFEVEEAAEAQAEPKDGSPEAPASPEREEVALSEYKTETYDDYKDETTIDDSIMDADSLWVDTQDDDRSIMTEQLETIPKEEKAEKEARRSSLEKHRKEKPFKTGRGRISTPERKVAKKEPSTVSRDEVRRKKAVYKKAELAKKTEVQAHSPSRKFILKPAIKYTRPTHLSCVKRKTTAAGGESALAPSVFKQAKDKVSDGVTKSPEKRSSLPRPSSILPPRRGVSGDRDENSFSLNSSISSSARRTTRSEPIRRAGKSGTSTPTTPGSTAITPGTPPSYSSRTPGTPGTPSYPRTPHTPGTPKSAILVPSEKKVAIIRTPPKSPATPKQLRLINQPLPDLKNVKSKIGSTDNIKYQPKGGQVQIVTKKIDLSHVTSKCGSLKNIRHRPGGGRVKIESVKLDFKEKAQAKVGSLDNAHHVPGGGNVKIDSQKLNFREHAKARVDHGAEIITQSPGRSSVASPRRLSNVSSSGSINLLESPQLATLAEDVTAALAKQGL

SEQ ID NO: 4 - protein, 582 aa, Homo sapiens
  misc_feature (1)..(582): myelin-associated glycoprotein
MIFLTALPLFWIMISASRGGHWGAWMPSSISAFEGTCVSIPCRFDFPDELRPAVVHGVWYFNSPYPKNYPPVVFKSRTQVVHESFQGRSRLLGDLGLRNCTLLLSNVSPELGGKYYFRGDLGGYNQYTFSEHSVLDIVNTPNIVVPPEVVAGTEVEVSCMVPDNCPELRPELSWLGHEGLGEPAVLGRLREDEGTWVQVSLLHFVPTREANGHRLGCQASFPNTTLQFEGYASMDVKYPPVIVEMNSSVEAIEGSHVSLLCGADSNPPPLLTWMRDGTVLREAVAESLLLELEEVTPAEDGVYACLAENAYGQDNRTVGLSVMYAPWKPTVNGTMVAVEGETVSILCSTQSNPDPILTIFKEKQILSTVIYESELQLELPAVSPEDDGEYWCVAENQYGQRATAFNLSVEFAPVLLLESHCAAARDTVQCLCVVKSNPEPSVAFELPSRNVTVNESEREFVYSERSGLVLTSILTLRGQAQAPPRVICTARNLYGAKSLELPFQGAHRLMWAKIGPVGAVVAFAILIAIVCYITQTRRKKNVTESPSFSAGDNPPVLFSSDFRISGAPEKYESKEVSTLESH

SEQ ID NO: 5 - protein, 313 aa, Homo sapiens
  misc_feature (1)..(313): calcium calmodulin dependent protein kinase
MHHHHHHSSGVDLGTENLYFQSMYQLFEELGKGAFSVVRRCVKVLAGQEYAAKIINTKKLSARDHQKLEREARICRLLKHPNIVRLHDSISEEGHHYLIFDLVTGGELFEDIVAREYYSEADASHCIQQILEAVLHCHQMGVVHRDLKPENLLLASKLKGAAVKLADFGLAIEVEGEQQAWFGFAGTPGYLSPEVLRKDPYGKPVDLWACGVILYILLVGYPPFWDEDQHRLYQQIKAGAYDFPSPEWDTVTPEAKDLINKMLTINPSKRITAAEALKHPWISHRSTVASCMHRQETVDCLKKFNARRKLKGA

SEQ ID NO: 6 - protein, 203 aa, Homo sapiens [sequence omitted]

SEQ ID NO: 7 - protein, 1020 aa, Homo sapiens
  misc_feature (1)..(1020): NFP heavy
MMSFGGADALLGAPFAPLHGGGSLHYALARKGGAGGTRSAAGSSSGFHSWTRTSVSSVSASPSRFRGAGAASSTDSLDTLSNGPEGCMVAVATSRSEKEQLQALNDRFAGYIDKVRQLEAHNRSLEGEAAALRQQQAGRSAMGELYEREVREMRGAVLRLGAARGQLRLEQEHLLEDIAHVRQRLDDEARQREEAEAAARALARFAQEAEAARVDLQKKAQALQEECGYLRRHHQEEVGELLGQIQGSGAAQAQMQAETRDALKCDVTSALREIRAQLEGHAVQSTLQSEEWFRVRLDRLSEAAKVNTDAMRSAQEEITEYRRQLQARTTELEALKSTKDSLERQRSELEDRHQADIASYQEAIQQLDAELRNTKWEMAAQLREYQDLLNVKMALDIEIAAYRKLLEGEECRIGFGPIPFSLPEGLPKIPSVSTHIKVKSEEKIKVVEKSEKETVIVEEQTEETQVTEEVTEEEEKEAKEEEGKEEEGGEEEEAEGGEEETKSPPAEEAASPEKEAKSPVKEEAKSPAEAKSPEKEEAKSPAEVKSPEKAKSPAKEEAKSPPEAKSPEKEEAKSPAEVKSPEKAKSPAKEEAKSPAEAKSPEKAKSPVKEEAKSPAEAKSPVKEEAKSPAEVKSPEKAKSPTKEEAKSPEKAKSPEKEEAKSPEKAKSPVKAEAKSPEKAKSPVKAEAKSPEKAKSPVKEEAKSPEKAKSPVKEEAKSPEKAKSPVKEEAKTPEKAKSPVKEEAKSPEKAKSPEKAKTLDVKSPEAKTPAKEEARSPADKFPEKAKSPVKEEVKSPEKAKSPLKEDAKAPEKEIPKKEEVKSPVKEEEKPQEVKVKEPPKKAEEEKAPATPKTEEKKDSKKEEAPKKEAPKPKVEEKKEPAVEKPKESKVEAKKEEAEDKKKVPTPEKEAPAKVEVKEDAKPKEKTEVAKKEPDDAKAKEPSKPAEKKEAAPEKKDTKEEKAKKPEEKPKTEAKAKEDDKTLSKEPSKPKAEKAEKSSSTDQKDSKPPEKATEDKAAKGK

SEQ ID NO: 8 - protein, 223 aa, Homo sapiens
  misc_feature (1)..(223): gamma-tubulin complex component 3
MTRSRREGDTGGTMEITEAALVRDILYVFQGIDGKNIKMNNTENCYKVEGKANLSRSLRDTAVRLSELGWLHNKIRRYTDQRSLDRSFGLVGQSFCAALHQELREYYRLLSVLHSQLQLEDDQGVNLGLESSLTLRRLLVWTYDPKIRLKTLAALVDHCQGRKGGELASAVHAYTKTGDPYMRSLVQHILSLVSHPVLSFLYRWIYDGELEDTYHEVRIGVRF

SEQ ID NO: 9 - protein, 92 aa, Homo sapiens
  misc_feature (1)..(92): protein S100-B
MSELEKAMVALIDVFHQYSGREGDKHKLKKSELKELINNELSHFLEEIKEQEVVDKVMETLDNDGDGECDFQEFMAFVAMVTTACHEFFEHE

SEQ ID NO: 10 - protein, 916 aa, Homo sapiens
  misc_feature (1)..(916): NFM
MSYTLDSLGNPSAYRRVTETRSSFSRVSGSPSSGFRSQSWSRGSPSTVSSSYKRSMLAPRLAYSSAMLSSAESSLDFSQSSSLLNGGSGPGGDYKLSRSNEKEQLQGLNDRFAGYIEKVHYLEQQNKEIEAEIQALRQKQASHAQLGDAYDQEIRELRATLEMVNHEKAQVQLDSDHLEEDIHRLKERFEEEARLRDDTEAAIRALRKDIEEASLVKVELDKKVQSLQDEVAFLRSNHEEEVADLLAQIQASHITVERKDYLKTDISTALKEIRSQLESHSDQNMHQAEEWFKCRYAKLTEAAEQNKEAIRSAKEEIAEYRRQLQSKSIELESVRGTKESLERQLSDIEERHNHDLSSYQDTIQQLENELRGTKWEMARHLREYQDLLNVKMALDIEIAAYRKLLEGEETRFSTFAGSITGPLYTHRPPITISSKIQKPKVEAPKLKVQHKFVEEIIEETKVEDEKSEMEEALTAITEELAVSMKEEKKEAAEEKEEEPEAEEEEVAAKKSPVKATAPEVKEEEGEKEEEEGQEEEEEEDEGAKSDQAEEGGSEKEGSSEKEEGEQEEGETEAEAEGEEAEAKEEKKVEEKSEEVATKEELVADAKVEKPEKAKSPVPKSPVEEKGKSPVPKSPVEEKGKSPVPKSPVEEKGKSPVPKSPVEEKGKSPVSKSPVEEKAKSPVPKSPVEEAKSKAEVGKGEQKEEEEKEVKEAPKEEKVEKKEEKPKDVPEKKKAESPVKEEAVAEVVTITKSVKVHLEKETKEEGKPLQQEKEKEKAGGEGGSEEEGSDKGAKGSRKEDIAVNGEVEGKEEVEQETKEKGSGREEEKGVVTNGLDLSPADEKKGGDKSEEKVVVTKTVEKITSEGGDGATKYITKSVTVTQKVEEHEETFEEKLVSTKKVEKVTSHAIVKEVTQSD

SEQ ID NO: 11 - protein, 543 aa, Homo sapiens
  misc_feature (1)..(543): NFL protein
MSSFSYEPYYSTSYKRRYVETPRVHISSVRSGYSTARSAYSSYSAPVSSSLSVRRSYSSSSGSLMPSLENLDLSQVAAISNDLKSIRTQEKAQLQDLNDRFASFIERVHELEQQNKVLEAELLVLRQKHSEPSRFRALYEQEIRDLRLAAEDATNEKQALQGEREGLEETLRNLQARYEEEVLSREDAEGRLMEARKGADEAALARAELEKRIDSLMDEISFLKKVHEEEIAELQAQIQYAQISVEMDVTKPDLSAALKDIRAQYEKLAAKNMQNAEEWFKSRFTVLTESAAKNTDAVRAAKDEVSESRRLLKAKTLEIEACRGMNEALEKQLQELEDKQNADISAMQDTINKLENELRTTKSEMARYLKEYQDLLNVKMALDIEIAAYRKLLEGEETRLSFTSVGSITSGYSQSSQVFGRSAYGGLQTSSYLMSTRSFPSYYTSHVQEEQIEVEETIEAAKAEEAKDEPPSEGEAEEEEKDKEEAEEEEAAEEEEAAKEESEEAKEEEEGGEGEEGEETKEAEEEEKKVEGAGEEQAAKKKD

SEQ ID NO: 12 - protein, 140 aa, Homo sapiens
  misc_feature (1)..(140): alpha-synuclein protein
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

(19) United States
(12) Patent Application Publication
(10) Pub. No.: US 2019/0137490 A1
(43) Pub. Date: May 9, 2019

(54) METHODS AND KITS FOR DIAGNOSING AND TREATING NERVOUS SYSTEM DISEASE OR INJURY

(71) Applicant: Duke University, Durham, NC (US)

(72) Inventor: Mohamed B. ABOU-DONIA, Durham, NC (US)

(73) Assignee: Duke University, Durham, NC (US)

(21) Appl. No.: 16/099,023

(22) PCT Filed: May 8, 2017
(86) PCT No.: PCT/US2017/031501

§ 371 Date: Nov. 5, 2018

Related U.S. Application Data
(60) Provisional application No. 62/332,689, filed on May 6, 2016.

(51) Int. Cl. G01N 33/564

(52) U.S. Cl. CPC G01N 33/564; G01N 2800/2835; G01N 2800/2871

(57) ABSTRACT

Methods of diagnosing nervous system injury or disease by measuring the level or presence of autoantibodies specific for and capable of binding to at least one protein selected from the group consisting of glial fibrillary acidic protein (GFAP), microtubule associated tau protein (Tau), microtubule associated protein-2 (MAP-2), myelin associated glycoprotein (MAG), calcium-calmodulin kinase II (CaM-KII), myelin basic protein (MBP), neurofilament triplet protein (NFP), NF200 (NFH), NF160 (NFM), NF68 (NFL), tubulin, α-synuclein (SNCA), and S100B protein in a sample from a subject. The methods also include measuring levels of autoantibodies specific for combinations of two or more of these proteins. Kits for performing the methods are also provided.

### CROSS-REFERENCE TO RELATED PATENT APPLICATIONS

The present application claims the benefit of priority of United States Provisional Patent Application No. 62/332,689, filed on May 6, 2016, the contents of which are incorporated herein by reference in its entirety.

### SEQUENCE LISTING

This application is being filed electronically via EFS-Web and includes an electronically submitted Sequence Listing in .txt format. The .txt file contains a sequence listing entitled “2017-05-08_5667-00399_Sequence_Listing_ST25.txt” created on May 8, 2017 and is 62,431 bytes in size. The Sequence Listing contained in this .txt file is part of the specification and is hereby incorporated by reference herein in its entirety.

### FIELD OF INVENTION

The invention generally relates to methods and kits for diagnosing brain injury. More specifically, the invention relates to use of autoantibody biomarkers to diagnose and treat nervous system diseases including, without limitation, Gulf War Illness, Parkinson's Disease, nervous system damage due to exposure to organophosphates (i.e, chlorpyrifos) or arsenic, stroke, autism, and Traumatic Brain Injury (TBI).

### INTRODUCTION

Nervous system injury may result from disease or following exposure to neurotoxic substances or head trauma. Such forms of both acute and chronic neurodegeneration can be very difficult to diagnosis in patients. Gulf War Illness (GWI) is one example of such injury. Approximately one third of the 697,000 United States military personnel who served in the Gulf War (GW) from August 1990 to June 1991 reported persistent symptoms during deployment and for many years after the war. These complex symptoms, known as GWI, include memory and attention problems, profound fatigue, chronic muscle and joint pain, severe headaches, persistent diarrhea, respiratory difficulties and skin rashes.

Epidemiological studies including brain imaging studies with GW veterans showed persistent signs and symptoms that were characteristic of CNS injury. There are, however, no validated objective diagnostic tests to identify acute or chronic sequelae of brain injury in this veteran group. Diagnosis of brain injury using cranial computed tomography (CT) scan and magnetic resonance imaging (MRI) techniques such as diffusion tensor imaging (DTI), have not been able to clinically diagnose veterans with GWI because there have been no proven cutoff values for volumetric or other imaging parameters that have been able to provide the required sensitivity/specificity needed for a diagnostic test marker. Although imaging studies have been able to show differences and altered CNS functioning between veterans with GWI and healthy controls, such studies have not yet been able to identify the groups diagnostically because of the significant overlap between the groups.

Similar to GWI, other nervous system conditions including, without limitation, Parkinson's Disease, stroke, autism, Traumatic Brain Injury (TBI), and nervous system damage due to exposure to organophosphates (i.e, chlorpyrifos) or arsenic are also difficult to diagnose. Without any validated diagnostic tests for these conditions, there is a significant need in the art to develop clinically available, simple and inexpensive biomarkers for detection and treatment of these conditions.

### SUMMARY

The invention generally relates to methods and kits for diagnosing nervous system injury or disease. More specifically, the invention relates to use of autoantibody biomarkers to diagnose nervous system conditions such as, without limitation, Gulf War Illness (GWI), Parkinson's Disease, stroke, autism, Traumatic Brain Injury (TBI), and nervous system damage due to exposure to organophosphates (i.e, chlorpyrifos) or arsenic.

In one aspect, methods for diagnosing nervous system injury or disease are provided. The methods may include obtaining a sample from a subject and measuring the level of at least one autoantibody capable of binding glial fibrillary acidic protein (GFAP), microtubule associated tau protein (Tau), microtubule associated protein-2 (MAP-2), myelin associated glycoprotein (MAG), calcium-calmodulin kinase II (CaM-KII), myelin basic protein (MBP), neurofilament triplet protein (NFP) including the neurofilament heavy, medium and light proteins (NFH or NF200; NFM or NF160; NFL or NF68), tubulin, α-synuclein (SNCA), S100B protein, or any combination thereof in the sample. Altered levels of autoantibodies are indicative of nervous system injury or disease and differential levels of the autoantibodies to the different proteins may be indicative of the specific type of nervous injury or disease. Thus, the methods may allow diagnosis of Gulf War Illness, Parkinson's disease, stroke, autism, traumatic brain injury (TBI), or exposure to toxins such as organophosphates, exhaust fumes or arsenic and/or be used to these differentiate between these conditions or differentiate these conditions from other types of nervous system injury or disease.

In another aspect, kits for diagnosing nervous system injury or disease are provided. The kits may include at least 2 proteins selected from the group consisting of GFAP, Tau, MAP-2, MAG, CaM-KII, MBP, NFP (NFH, NFM, NFL), tubulin, α-synuclein (SNCA), and S100B protein.

### BRIEF DESCRIPTION OF THE DRAWINGS

FIG. 1 shows a representative sample of Western blot gels from three cases showing that the majority of GWI serum reacted intensely to neural proteins (FIG. 1B), while most control serum showed a weak or no reaction (FIG. 1A).

FIG. 2 shows mean autoantibodies against neural proteins from cases and controls expressed in mean optical density units.

FIG. 3 shows fold increase of autoantibodies against neural proteins from cases relative to controls.

FIG. 4 shows the levels of autoantibodies of neural proteins of GWI cases and of controls expressed as optical density units.

FIG. 5 shows paired correlations of Tau and MBP optical density levels in cases relative to controls.

FIG. 6A shows tubulin levels were higher than all controls in 12/20 cases. FIG. 6B shows GFAP levels were higher than all controls in 20/20 cases. FIG. 6C shows Tau levels were higher than all controls in 17/20 cases. FIG. 6D shows MAP levels were higher than all controls in 15/20 cases. FIG. 6E shows MBP levels were higher than all controls in 12/20 cases. FIG. 6F shows NFP levels were higher than all controls in 10/20 cases. FIG. 6G shows MAG levels were higher than all controls in 15/20 cases. FIG. 6H shows CAMKII levels were higher than all controls in 16/20 cases. FIG. 6I shows S100B levels overlap with cases and controls.

FIG. 7 shows the levels of autoantibodies in the serum of a Parkinson's Disease patient. The levels of autoantibodies against neural proteins were, in descending order: Tau, GFAP, NFP, MBP, Tubulin, MAP-2, S-100B.

FIG. 8A shows the detection of autoantibodies to neurofilament proteins by Western blot in a patient exposed to the organophoshorus insecticide—Chlorpyrifos. P=standard proteins; 1=patient (5 year old), 2=brother (6 year old), 3=brother (9 year old), 4=father, 5=mother. FIG. 8B shows the quantification of autoantibodies to either NFH (NF200) or NFM (NF160) in the tested patients as measured by densitometry units.

FIG. 9 shows the relative levels of autoantibodies to neural proteins (NFP, Tau, Tubulin, MBP, MAP-2, GFAP, and S-100B) in the serum of a group of 34 pilots and flight attendants that had allegedly been exposed to air emissions (engine oil contaminants, i.e., gaseous, vapor, and particulate constituents of pyrolyzed engine oil) in the unfiltered ventilation air supply that is extracted from either the aircraft engines or auxiliary power unit (APU). These levels were compared to a matched group of 12 healthy controls.

FIG. 10 shows the relative levels of autoantibodies to neural proteins (NFL, NFM, NFH, MAP-2, and Tau) in the serum of a group of 14 subjects from a highly Arsenic-contaminated village of Mianpur in Bangladesh. These levels were compared to a matched group of 8 healthy controls.

FIG. 11 shows the relative levels of autoantibodies to neural proteins (NFP, Tau, Tubulin, MBP, MAG, MAP-2, GFAP, and S-100B) in the serum of a group of subjects that had a stroke. These levels were compared to a matched group of healthy controls.

FIG. 12 shows the relative levels of autoantibodies to neural proteins (NFP, Tau, Tubulin, MBP, MAP-2, GFAP, and S-100B) in the serum of a group of 10 subjects with Traumatic Brain Injury (TBI). These levels were compared to a matched group of 8 healthy controls.

FIG. 13 shows the detection by Western Blot of circulating autoantibodies to a panel of proteins associated with the nervous system in sera of three control children and three control mothers (FIG. 13A) and three autistic children and three autistic mothers (FIG. 13B).

FIG. 14 shows the levels of autoantibodies to neural and glial proteins from the serum of children with autism as compared to normal control children (FIG. 14A) and the mothers of the autistic children (FIG. 14B).

### DETAILED DESCRIPTION

The present invention generally relates to the discovery of objective biomarkers of nervous system injury or disease and, in particular, biomarkers important for diagnosing conditions such as, without limitation, Gulf War Illness (GWI), Parkinson's Disease, nervous system damage due to exposure to organophosphates (i.e, chlorpyrifos), exhaust fumes, or arsenic, stroke, autism, and Traumatic Brain Injury (TBI). Without being limited by theory, the present work suggests that various nervous system conditions lead to blood brain barrier leakage of specific neuralproteins into circulation, with subsequent formation of particular levels of autoantibodies (AB) against these proteins. The inventors have discovered that such autoantibodies can be quantified and used as sensitive biomarkers for the assessment of various nervous system diseases and forms of injury.

In non-limiting Example 1, the inventors measured the levels of circulating IgG-class autoantibodies in sera from GWI subjects and symptomatic controls against several brain proteins including neurofilament triplet proteins (NFP), tubulin, microtubule associated protein-tau (tau proteins), microtubule associated protein-2 (MAP-2), calcium/calmodulin Kinase II (CaMKII), myelin basic protein (MBP), myelin associated glycoprotein (MAG), glial fibrillary acidic protein (GFAP) and glial S100B protein. Significantly elevated levels of autoantibodies against several of these neurotypic- and gliotypic-specific proteins were found in sera from a sample of veterans with GWI as compared to non-veteran symptomatic controls.

Likewise, in non-limiting Examples 2-8, significantly elevated levels of autoantibodies against several neural proteins were found in sera from subjects with various nervous system injuries and/or diseases including Parkinson's Disease, stroke, autism, Traumatic Brain Injury (TBI), and nervous system damage due to exposure to organophosphates (i.e, chlorpyrifos), exhaust fumes or arsenic.

The identification and use of the autoantibody biomarkers shown here have an important diagnostic value. The relative non-invasiveness, low cost, and dynamism of autoantibodies make a diagnostic test of various nervous system conditions well-suited for incorporation into routine health care. With such a diagnostic test, accessible early screening methods can be established so that subjects will be better positioned to avail themselves of effective therapies. The autoantibody biomarkers identified here may also be used for “fingerprinting” neurotoxicity induced by exposure to particular neurotoxicants, such as organophosphates, arsenic or exhaust fumes.

In some embodiments, the methods provided herein may include obtaining a sample from a subject and measuring the level of at least one autoantibody capable of binding a neural protein in the sample. The methods may be used to diagnose nervous system injury or disease in a subject and may further include comparing the level of the at least one autoantibody in the sample to a reference level of the autoantibody and/or diagnosing the subject with nervous system injury or disease if the level of the at least one autoantibody is elevated as compared to the reference level. Altered levels, in particular increased levels, of autoantibodies are indicative of nervous system injury or disease and differential levels of the autoantibodies to the different proteins may be indicative of the specific type of nervous system injury or disease.

As used herein, a “neural protein” may include any protein that is specifically expressed in or on a cell within the nervous system. The neural protein may be from any type of cell within the nervous system including, without limitation, neurons and glial cells.

The methods provided herein may include obtaining a sample from a subject and measuring the level of at least one autoantibody capable of binding glial fibrillary acidic protein (GFAP), microtubule associated tau protein (Tau), microtubule associated protein-2 (MAP-2), myelin associated glycoprotein (MAG), calcium-calmodulin kinase II (CaM-KII), myelin basic protein (MBP), neurofilament triplet protein (NFP) including NFH, NFM and NFL, tubulin, α-synuclein (SNCA), S100B protein, or any combination thereof in the sample. The methods may be used to diagnose nervous system injury or disease in a subject and may further include comparing the level of the at least one autoantibody in the sample to a reference level of the autoantibody and/or diagnosing the subject with nervous system injury or disease if the level of the at least one autoantibody is elevated as compared to the reference level. Altered levels of autoantibodies are indicative of nervous system injury or disease and differential levels of the autoantibodies to the different proteins may be indicative of the specific type of nervous system injury or disease. Thus, the methods may allow diagnosis of, for example, Gulf War Illness, Parkinson's Disease, stroke, autism, Traumatic Brain Injury (TBI), and nervous system damage due to exposure to toxic agents such as organophosphates (i.e, chlorpyrifos), exhaust fumes, airline toxins, or arsenic and/or be used to differentiate these conditions from other types of nervous system injury or disease. For example autoantibodies to S100B are generally elevated in subjects after stroke or traumatic brain injury, but are generally not elevated in subjects with Gulf War Illness. Thus the methods may be used for differential diagnosis of these brain injuries. Notably such a differential diagnosis may provide medical professionals with distinct treatment options based on the type of nervous system injury or disease.

Methods of detecting autoantibodies capable of binding glial fibrillary acidic protein (GFAP), microtubule associated tau protein (Tau), microtubule associated protein-2 (MAP-2), myelin associated glycoprotein (MAG), calcium-calmodulin kinase II (CaM-KII), myelin basic protein (MBP), neurofilament triplet protein (NFP) including NFH, NFM and NFL, tubulin, α-synuclein (SNCA), S100B protein or combinations thereof. The methods include obtaining a sample from the subject and determining if the sample contains auto-antibodies to any of the indicated proteins. The methods may include detecting antibodies to at least 5 of the proteins in the list above. The methods may include detecting antibodies to at least 7 of the proteins in the list above. The methods may include detecting antibodies to all of the proteins in the list above. The autoantibodies if present are indicative of brain injury and may be used to determine the type of brain injury or disease. Thus the methods may be used to diagnose whether the subject has Gulf War Illness, Parkinson's Disease, stroke, autism, Traumatic Brain Injury (TBI), and nervous system damage due to exposure to toxic agents such as organophosphates (i.e, chlorpyrifos), exhaust fumes, airline toxins, or arsenic. In addition subjects diagnosed with one of the diseases or injuries may be treated with immunosuppresives or analgesics or other pharmaceuticals to treat the disease.

As used herein, “nervous system injury or disease” refers to any injury or disease of the nervous system including, without limitation, chronic or acute injury that may result from disease or following exposure of the nervous system to neurotoxic substances or trauma. In some embodiments, the nervous system injury or disease comprises Gulf War Illness (GWI), Parkinson's Disease, stroke, autism, Traumatic Brain Injury (TBI), or nervous system damage due to exposure to toxic agents such as organophosphates (i.e, chlorpyrifos), exhaust fumes or arsenic.

GWI refers to the complex group of symptoms experienced by thousands of Gulf War military personnel during deployment and for many years after the war. The subjects may also include personnel who spent time in the Persian Gulf region during any of the military engagements in that region such as military support personnel or private contractors. These complex symptoms may include, without limitation, memory and attention problems, profound fatigue, chronic muscle and joint pain, severe headaches, persistent diarrhea, respiratory difficulties and skin rashes.

As used herein, the term “subject” and “patient” are used interchangeably and refer to both human and non-human animals. The term “non-human animals” as used in the disclosure includes all vertebrates, e.g., mammals and non-mammals, such as non-human primates, sheep, dog, cat, horse, cow, chickens, amphibians, reptiles, and the like. Suitably, the subject is a human. In some embodiments, the human subject has served in the Gulf War and/or may be suspected of being exposed to toxic substances such as, without limitation, pyridostigmine bromide (PB), sarin, soman, DEET (insect repellent), permethrin, and/or organophosphates such as chlorpyrifos, exhaust fumes or chemicals used in the airline industry or arsenic.

The methods of the present invention may include obtaining a sample from a subject. The sample may or may not include cells. In particular, the methods described herein may be performed without requiring a tissue sample or biopsy. “Sample” is intended to include any sampling of cells, tissues, or bodily fluids in which a level of an autoantibody can be detected. Examples of such samples include, but are not limited to, blood, serum, urine, synovial fluid, saliva, or any other bodily secretion or derivative thereof. Blood can include whole blood, plasma (citrate, EDTA, heparin), serum, or any derivative of blood. Samples may be obtained from a patient by a variety of techniques available to those skilled in the art. Methods for collecting various samples are well known in the art. In some embodiments, the sample is serum or plasma.

The present methods may include measuring the level of at least one autoantibody in the sample. An “autoantibody” is an antibody generated in a subject that is capable of binding a protein found in the subject. The autoantibody may be any one of the classes of antibodies including, without limitation, IgA, IgG, IgM, IgE or IgD immunoglobulins. Suitably, the autoantibody is an IgG immunoglobulin.

Any methods available in the art for measuring the level of autoantibodies are encompassed herein. For example, the level of an autoantibody in a sample may be measured using the antigenic protein the autoantibody is specific for. For example, the autoantibodies may be detected by incubation of the sample with the protein bound or cross-linked to a solid support such as in an ELISA or Western blot followed by detection with a secondary antibody-link3ed to a detectable label. Such antibodies are commercially available. “Measuring the level of” is intended to mean determining the quantity or presence of an autoantibody in a sample. Thus, “measuring the level of” encompasses instances where an autoantibody is determined not to be detectable due to failure to be produced, or due to production below the detection limit of the assay; “measuring the level of” also encompasses low, normal and high levels of detection. Thus a relative level or presence or absence can be determined when measuring a level. This may include determining if the sample has any autoantibodies to a particular protein in the list of neural proteins provided herein.

Methods suitable for “measuring the level of” autoantibodies are known to those of skill in the art and include, but are not limited to, western blot, ELISA, immunofluorescence, FACS analysis, dot blot, magnetic immunoassays, mass spectroscopy, gel electrophoresis, antigenic protein microarrays and non-antigenic protein-based microarrays or combinations of these methods.

The autoantibodies of the present invention may be capable of binding neural proteins. The neural proteins may include glial fibrillary acidic protein (GFAP), microtubule associated tau protein (Tau), microtubule associated protein-2 (MAP-2), myelin associated glycoprotein (MAG), calcium-calmodulin kinasell (CaM-KII), myelin basic protein (MBP), neurofilament triplet protein (NFP) including NFH, NFM, or NFL, tubulin, α-synuclein (SNCA), S100B protein, or any combination thereof in the sample. In some embodiments, the level of autoantibodies capable of binding at least 2, 3, 4, 5, 6, 7, 8, 9, 10, 11, or 12 of these proteins are measured.

The autoantibodies of the present invention may be capable of binding GFAP, Tau, MAP-2, MAG, CaM-KII, S100B, or any combination thereof in the sample. In some embodiments, the level of autoantibodies capable of binding at least 2, 3, 4, or 5 of these proteins are measured. In some embodiments, the level of autoantibodies capable of binding GFAP, Tau, MAP-2, MAG, CaM-KII, and S100B proteins are all measured. The levels of the autoantibodies may be determined individually or by measuring a total number of autoantibodies capable of recognizing a set of two or more proteins. For example, when measuring a total number of autoantibodies capable of recognizing a set of two or more proteins, the two or more proteins may be combined and adapted for a particular assay including, but are not limited to, western blot, ELISA, immunofluorescence, FACS analysis, dot blot, magnetic immunoassays, mass spectroscopy, gel electrophoresis, antigenic protein microarrays and non-antigenic protein-based microarrays or combinations of these methods.

The neural proteins disclosed herein represent various anatomical regions of the neuron or glial cell with distinct functions. As used herein, a “polypeptide” or “protein” or “peptide” may be used interchangeably to refer to a polymer of amino acids. A “protein” as contemplated herein typically comprises a polymer of naturally occurring amino acids (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine). The proteins described herein are known to those of skill in the art as delineated below and are available as commercial proteins. The nucleotide and protein sequences are also publicly available. The proteins for use in the methods may be obtained from commercial sources or may be produced for use in the methods by any means available to those of skill in the art. Portions of the full-length proteins may also be used to detect autoantibodies directed to these portions of the full-length proteins. As those skilled in the art are aware antibodies generally recognize short epitopes of between 6 and 10 amino acids that may be linear or conformational and may include recognition of various modifications to the proteins including but not limited to methylation, acylation and addition of sugar moieties. Thus small portions of the proteins may be used in the methods described herein and the proteins may contain these modifications or not. Proteins containing mixtures of alleles of the proteins may also be used. As noted in the examples, bovine or other mammalian proteins may be used to detect human autoantibodies.

Glial fibrillary acidic protein (GFAP) is expressed almost exclusively in astrocytes, where it is induced by neural injury and released upon disintegration of the astrocyte cytoskeleton. GFAP plays an essential role in maintaining shape and motility of astrocytic processes and contribute to white matter architecture, myelination and blood brain barrier (BBB) integrity. The GFAP protein may comprise the human GFAP protein sequence of SEQ ID NO: 1 or a protein having at least 60%, 70%, 75%, 80%, 85%, 90%, 95%, or 98% sequence identity to SEQ ID NO: 1.

Microtubule associated tau protein (Tau) is a normal axonal protein that is involved in the stabilization and assembly of axonal microtubules. The Tau protein may comprise the human Tau protein sequence of SEQ ID NO: 2 or a protein having at least 60%, 70%, 75%, 80%, 85%, 90%, 95%, or 98% sequence identity to SEQ ID NO: 2.

Microtubule associated protein-2 (MAP-2) is found in dendritic compartments of neurons. The MAP-2 protein may comprise the human MAP-2 protein sequence of SEQ ID NO: 3 or a protein having at least 60%, 70%, 75%, 80%, 85%, 90%, 95%, or 98% sequence identity to SEQ ID NO: 3.

Myelin Associated Glycoprotein (MAG) is selectively localized in periaxonal Schwann cell and oligodendroglial membranes of myelin sheaths, suggesting that it functions in glia-axon interactions in both the PNS and CNS. The MAG protein may comprise the human MAG protein sequence of SEQ ID NO: 4 or a protein having at least 60%, 70%, 75%, 80%, 85%, 90%, 95%, or 98% sequence identity to SEQ ID NO: 4.

Calcium-calmodulin kinase II (CaM-KII) phosphorylates cytoskeletal proteins, such as MAP-2, tau, tubulin. CaMKII accounts for 12% of all proteins in the brain. CaMKII has the ability to coordinate and transduce upstream Ca²⁺ and reactive oxygen species (ROS) signals into physiological and pathophysiological downstream responses in the nervous system and cardiovascular biology and disease. The CaM-KII protein may comprise the human CaM-KII protein sequence of SEQ ID NO: 5 or a protein having at least 60%, 70%, 75%, 80%, 85%, 90%, 95%, or 98% sequence identity to SEQ ID NO: 5.

Myelin Basic Protein (MBP) is an abundant myelin membrane proteolipid produced by oligodendroglia in the CNS and Schwann cells in PNS and may confirm the clinical assessment of neurodegenerative disorders such as multiple sclerosis and stroke. The MBP protein may comprise the human MBP protein sequence of SEQ ID NO: 6 or a protein having at least 60%, 70%, 75%, 80%, 85%, 90%, 95%, or 98% sequence identity to SEQ ID NO: 6.

Neurofilament triplet protein (NFP) refers to all or any one of the three major neurofilament subunits that are a major component of the neuronal cytoskeleton. The NFP protein may comprise the human NFP protein sequence of SEQ ID NO: 7 which may also be called NFH, NF200 or the heavy chain or a protein having at least 60%, 70%, 75%, 80%, 85%, 90%, 95%, or 98% sequence identity to SEQ ID NO: 7. The other two neurofilament proteins NFM (NF160; SEQ ID NO: 10) and NFL (NF68; SEQ ID NO: 11) are also included as are proteins having at least 60%, 70%, 75%, 80%, 85%, 90%, 95%, or 98% sequence identity to SEQ ID NOs: 10 or 11.

Tubulin is the major component of microtubules and is responsible for axonal migration and longitudinal growth and is involved in axonal transport. Although tubulin is present in virtually all eukaryotic cells, the most abundant source is the vertebrate brain, where it consists of approximately 10-20% of its total soluble protein. The tubulin protein may comprise the human tubulin protein sequence of SEQ ID NO: 8 or a protein having at least 60%, 70%, 75%, 80%, 85%, 90%, 95%, or 98% sequence identity to SEQ ID NO: 8.

S100B protein exerts both detrimental and neurotrophic effects, depending on its concentration in brain tissues. For example, after release, S-100B acts as a trophic factor for serotoninergic neurons, and plays a role in axonal growth and synaptogensis during development. The S100B protein may comprise the human S100B protein sequence of SEQ ID NO: 9 or a protein having at least 60%, 70%, 75%, 80%, 85%, 90%, 95%, or 98% sequence identity to SEQ ID NO: 9.

α-synuclein (SNCA) is abundant in the brain and found mainly at the tips of nerve cells at presynaptic terminals. The protein may play a role in Parkinson's and Alzheimer's Disease pathogenesis. The SNCA protein may comprise the human SNCA protein sequence of SEQ ID NO: 12 or a protein having at least 60%, 70%, 75%, 80%, 85%, 90%, 95%, or 98% sequence identity to SEQ ID NO: 12.

Many of the proteins described herein are involved in axonal structure and function and are released as products of neural degeneration of various regions of the neuron. MAP-2 is present in the dendrites; CaMKII, tau, tubulin, and neurofilament proteins are located in the axon; myelin basic protein (MBP) and myelin associated glycoprotein (MAG) are an integral part of myelin. Furthermore, the central nervous system-specific glial protein, GFAP and S-100B are secreted by astrocytes after neuronal injury. As shown here, following exposure to neurotoxic substances or trauma these neuronal and glial proteins are released and once in circulation, activated lymphocyte cells lead to the formation of autoantibodies against these proteins. Normally these proteins are only found in the brain and are protected from the immune system, but when nervous system injury or disease occurs these proteins may escape through the blood brain barrier and be exposed to the immune system for the first time resulting in generation of autoantibodies.

The proteins used in the methods of detecting autoantibodies provided herein may be full-length polypeptides or may be fragments of the full-length polypeptide. As used herein, a “fragment” is a portion of an amino acid sequence which is identical in sequence to but shorter in length than a reference sequence. A fragment may comprise up to the entire length of the reference sequence, minus at least one amino acid residue. In some embodiments, a fragment may comprise at least 5, 10, 15, 20, 25, 30, 40, 50, 60, 70, 80, 90, 100, 150, 250, or 500 contiguous amino acid residues of a reference protein. Fragments may be preferentially selected from certain regions of a molecule. The term “at least a fragment” encompasses the full length polypeptide. A fragment of a protein may comprise or consist essentially of a contiguous portion of an amino acid sequence of the full-length protein. A fragment may include an N-terminal truncation, a C-terminal truncation, or both truncations relative to the full-length wild-type protein. Suitably, the fragments are immunogenic.

In the present methods, the level of the autoantibody in the sample from the subject may be compared to a reference level of the autoantibody. The reference level may be determined empirically such as illustrated in the Examples, by comparison to the levels found in a set of samples from subjects with known clinical outcomes or known to not have nervous system injury or disease. Alternatively, the reference level may be a level of the autoantibody found in samples, such as serum samples, which becomes a standard and can be used as a predictor for new samples. The level of the autoantibody in the sample from the subject may be increased as compared to the reference level.

The predictive methods described herein may be combined to provide increased significance of the results. For example, the levels of multiple autoantibodies may be determined in a sample from the subject and the results may have additional statistical or predictive power via the combination. The levels may be compared to the reference levels and a diagnosis or a prediction of nervous system injury or disease made.

The present methods may further include administering immunosuppressants or anti-inflammatory agents or anti-pain agents or combinations thereof to the subject if the subject is diagnosed with nervous system injury or disease. Immunosuppressants include, but are not limited to, prednisone, azathioprine, cyclosporine, basiliximab, daclizumab, muromonab, corticosteroids, glucocorticoids, methotrexate, cyclophosphamide, prednisolone, methylprednisolone, a-methapred, Medrol, Depo-Medrol, Solu-medrol, cotolone, prednicot, sterapred, prelone, veripred, millipred, orapred, flo-pred, sterapred, pedipred, and methylpred. Anti-inflammatory agents include, but are not limited to, the NSAIDS (non-steroidal anti-inflammatory agents) such as aspirin, ibuprofen, naproxen, celecoxib and many others and also includes steroidal anti-inflammatory agents. Suitable anti-pain agents include, without limitation, non-opioid analgesics (e.g., acetaminophen), opioid analgesics, and co-analgesics.

The present methods may further include administering therapeutic agents used to treat the specific nervous system injury or diseased diagnosed. For example, subjects diagnosed with Parkinson's Disease may be treated with therapeutic agents including, without limitation, L-Dopa (or other forms of dopamine such as carbidopa-levodopa), dopamine agonists, MAO-B inhibitors, Catechol-O-methyltransferase (COMT) inhibitors, anticholinergics, or amantadines. Subjects diagnosed with stroke may be treated with therapeutic agents including, without limitation, NSAIDS (non-steroidal anti-inflammatory agents) such as aspirin, tissue plasminogen activator (TPA), warfarin, or clopidogrel.

As noted in the subsequent discussion those of skill in the art will appreciate that assays (kits and methods of using them) can be developed to differentially diagnose various types of nervous system injury or disease using the proteins described herein and specifically screening for the presence and levels of autoantibodies to these proteins in samples from subjects. The methods may include screening the levels of autoantibodies to two, three, four or more of the listed proteins. The various nervous system injuries or diseases are shown herein to display a “fingerprint” of autoantibodies to a specific set of proteins (or lack of autoantibodies) which can be used for diagnosis and treatment of the underlying injury or disease. In one embodiment, autoantibodies specific for at least two, three, four, five, six, seven or eight of the proteins selected from the group consisting of GFAP, Tau, tubulin, MAP, MBP, NFP, MAG, CAMKII are measured and if the levels are increased as compared to the reference levels than the subject is diagnosed with Gulf War Illness. The levels may be increased by two fold, three fold or more relative to the reference levels. The level of S100B specific autoantibodies in the sample may also be measured, if the levels of autoantibodies are increased less than two fold it is consistent with and indicative of a diagnosis of Gulf War Illness.

In another embodiment, autoantibodies specific for at least two, three or all four of MBP, MAP-2, GFAP or S100B are increased as compared to the reference levels of autoantibodies then the subject is diagnosed with TBI. The increase may be two, three four or even five fold or more as compared to the reference level.

In another embodiment, autoantibodies specific for at least two, three, four, five of NFP, Tau, tubulin, MBP and GFAP are measured and if the levels of autoantibodies are increased as compared to levels in the reference, then the subject is diagnosed with Parkinson's Disease. The levels may be increased by two fold, three fold, four fold five fold or more as compared to reference levels. The level of S100B specific autoantibodies in the sample may also be measured, if the levels of autoantibodies are increased less than four fold, three fold, or two fold it is consistent with and indicative of a diagnosis of Parkinson's disease.

In another embodiment, autoantibodies specific for at least one or both of NFM and NFH are measured and if the levels of autoantibodies are increased in the subject as compared to the reference levels, then the subject is diagnosed with organophosphate exposure. The levels may be increased by two, three, four, five fold or more relative to the reference.

In another embodiment, autoantibodies specific for at least two, three, four, five or all six of NFP, Tau, tubulin, MBP, MAP-2 and GFAP are measured and if the levels of autoantibodies are increased in the subject as compared to the reference levels, then the subject is diagnosed with exposure to toxic fumes. The levels may be increased by two, three, four fold or more relative to the reference. The level of S100B specific autoantibodies in the sample may also be measured, if the levels of autoantibodies are increased less than two fold it is consistent with and indicative of a diagnosis of exposure to toxic fumes such as airline exhaust.

In another embodiment, autoantibodies specific for at least one or both of NFL or Tau are measured and if the levels of autoantibodies are increased in the subject as compared to the reference levels, then the subject is diagnosed with exposure to arsenic. The levels may be increased by two, three, four fold or more relative to the reference.

In another embodiment, autoantibodies specific for at least two, three, four or five of NFP, tubulin, MBP, MAG, S100B and GFAP are measured and if the levels of autoantibodies are increased in the subject as compared to the reference levels, then the subject is diagnosed with stroke. The levels may be increased by two, three, four, five fold or more relative to the reference.

In another embodiment, autoantibodies specific for at least two, three, four, five, six or more of NFP, MBP, MAP-2, MAG, a-synuclein, Tau, S100B and GFAP are measured and if the levels of autoantibodies are increased in the subject or in the subject's mother as compared to the reference levels, then the subject is diagnosed with autism. The levels may be increased by two, three, four, five fold or more relative to the reference. The mothers of autistic children may also be assessed. Autoantibodies specific for at least one, two three of all four of GFAP, MAP2, NFP, MBP in samples from mothers which are increased by at least two fold relative to reference levels are indicative of a child with autism.

Kits for diagnosing nervous system injury or disease are provided. The kits may include at least 2, 3, 4, 5, 6, 7, 8, 9, 10, 11, or 12 proteins selected from the group consisting of GFAP, Tau, MAP-2, MAG, CaM-KII, MBP, NFP (NFH, NFM, NFL), tubulin, α-synuclein (SNCA), and S100B protein. The kits may also include at least 2, 3, 4, or 5 proteins selected from the group consisting of GFAP, Tau, MAP-2, MAG, CaM-KII, and S100B. In some embodiments, the kits include GFAP, Tau, MAP-2, MAG, CaM-KII, and S100B proteins.

The kits of the present disclosure may further include an anti-subject antibody, such as an anti-IgG, capable of binding an autoantibody and conjugated to a detectable label. The anti-subject antibody may have been raised in any vertebrate species including, without limitation, primates, mice, rats, goats, chickens, rabbits, donkeys, and the like and may be specific to immunoglobulins such as, without limitation IgA, IgG, IgM, IgE or IgD immunoglobulins or may be pan-specific. Preferably, the anti-subject antibody is an anti-human IgG antibody conjugated to a detectable label. The detectable label may be any label that may be detected using laboratory methods that does not interfere substantially with binding of the anti-subject antibody to the autoantibody. Such antibodies are available commercially and can be made by those of skill in the art. Suitable detectable labels include enzymes, such as horseradish peroxidase (HRP), fluorescent labels, and radioactive labels.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference in their entirety, unless explicitly indicated otherwise. The present disclosure shall control in the event there are any disparities between any definitions and/or description found in the cited references.

Unless otherwise specified or indicated by context, the terms “a”, “an”, and “the” mean “one or more.” For example, “a protein” or “an RNA” should be interpreted to mean “one or more proteins” or “one or more RNAs,” respectively.

The following examples are meant only to be illustrative and are not meant as limitations on the scope of the invention or of the appended claims.

### EXAMPLES

### Example 1

Screening for Novel Central Nervous System Biomarkers in Veterans with Gulf War Illness

Gulf War illness (GWI) is primarily diagnosed by symptom report; objective biomarkers are needed that distinguish those with GWI. Prior chemical exposures during deployment have been associated in epidemiologic studies with altered central nervous system functioning in veterans with GWI. Previous studies from our group have demonstrated the presence of autoantibodies to essential neuronal and glial proteins in patients with brain injury and autoantibodies have been identified as candidate objective markers that may distinguish GWI. Here, we screened the serum of 20 veterans with GWI and 10 non-veteran symptomatic (low back pain) controls for the presence of such autoantibodies using Western blot analysis against the following proteins: neurofilament triplet proteins (NFP), tubulin, microtubule associated tau proteins (Tau), microtubule associated protein-2 (MAP-2), myelin basic protein (MBP), myelin associated glycoprotein (MAG), glial fibrillary acidic protein (GFAP), calcium-calmodulin kinase II (CaMKII) and glial 5-100B protein. Serum reactivity was measured as arbitrary chemiluminescence units. As a group, veterans with GWI had statistically significantly higher levels of autoantibody reactivity in all proteins examined except S-100B. Fold increase of the cases relative to controls in descending order were: CaMKII 9.27, GFAP 6.60, Tau 4.83, Tubulin 4.41, MAG 3.60, MBP 2.50, NFP 2.45, MAP-2 2.30, S-100B 1.03. These results confirm the continuing presence of neuronal injury/gliosis in these veterans and are in agreement with the recent reports indicating that 25 years after the war, the health of veterans with GWI is not improving and may be getting worse. Such serum autoantibodies may prove useful as biomarkers of GWI, upon validation of the findings using larger cohorts.

Approximately one third of the 697,000 US military personnel who served in the Gulf War (GW) from August 1990 to June 1991, have reported persistent symptoms for many years after the war. This complex of symptoms, known as Gulf War Illness (GWI), include memory and attention problems, profound fatigue, chronic muscle and joint pain, severe headaches, persistent diarrhea, respiratory difficulties and skin rashes. GWI is primarily diagnosed by symptom report and no validated objective diagnostic biomarkers currently exist that fully segregate cases from controls. This study was designed to identify objective central nervous system (CNS) biomarkers of GWI using clues from prior clinical studies with GW veterans and from animal studies that modeled chemical exposures experienced by GW veterans.

Clinical studies have reported impaired cognitive functioning and reduced MRI volume and altered white matter microstructural integrity in organophosphate (OP) pesticide, sarin nerve agent and pyridogstigmine bromide (PB) anti-nerve gas pill-exposed GWveteran cohorts (White et al., 2016; Sullivan et al., 2013; Chao et al., 2010; Heaton et al., 2007; Proctor et al., 2006; Sullivan et al., 2003). Animal studies demonstrated that exposure to higher doses of the prophylaxis pill pyridostigmine bromide (PB), the insect repellent, DEET, and the insecticide permethrin and/or chlorpyrifos led to significant brain damage in animal models of GWI (Abou-Donia et al., 1996a,b). Further studies using 60 days of subchronic dermal exposure to DEET and permethrin, alone or in combination, at dose levels approximately equivalent to the exposures that occurred during the Gulf War in a rat-model of GWI, caused the following: (1) a diffuse neuronal cell death in the motor cortex, the different subfields of the hippocampal formation, and the Purkinje cell layer of the cerebellum, accompanied by sensorimotor deficits; (2) significant reduction of MAP-2-positive immunoreactive structures indicating atypical expression of MAP-2 in dendrites of surviving neurons, within the cerebral cortex and the hippocampus that was characterized by a beaded, disrupted, or wavy appearance; (3) a significant upregulation of GFAP-positive expression in structures in the CA3 subfield of the hippocampus, the motor cortex and the dentate gyrus (Abdel-Rahman et al., 2001, 2002a,b, 2004a,b; Abou-Donia et al., 2000, 2001, 2002, 2004; Terry et al., 2003). Similar results were exhibited in animals treated with sarin alone or accompanied by cited-above chemicals, with and without stress (Abdel-Rahman et al., 2004a).

The cytoarchitecture of the CNS is maintained by a complex cellular milieu that involves neuronal and glial cells that must maintain proper communication in order to function properly (Abou-Donia and Lapadula, 1990; McMurray, 2000). CaMKII phosphorylates cytoskeletal proteins, such as MAP-2, tau and tubulin. CaMKII accounts for 12% of all proteins in the brain. CaMKII has the ability to coordinate and transduce upstream Ca and reactive oxygen species (ROS) signals into physiological and pathophysiological downstream responses in the nervous system and cardiovascular biology and disease (Abou-Donia, 1995; Erickson et al., 2011). Tubulin, the major component of microtubules, is responsible for axonal migration and longitudinal growth and is involved in axonal transport. Although tubulin is present in virtually all eukaryotic cells, the most abundant source is the vertebrate brain, where it consists of approximately 10-20% of its total soluble protein (McMurray, 2000). Microtubule-Associated Protein-2 (MAP-2) is found in dendritic compartments of neurons. A loss of MAP-2, is a reliable indication of irreversible neuropathology and is a sensitive marker of seizure-related brain damage (Ballough et al., 1995). Tau Protein, a normal axonal protein, is involved in stabilization and assembly of axonal microtubules. Levels of tau proteins are elevated in the cerebrospinal fluid (CSF) and serum following TBI (Liliang et al., 2011) and increased levels have been used for diagnosis of Alzheimer's disease. Myelin basic protein (MBP) is an abundant myelin membrane proteolipid produced by oligodendroglia in the CNS and Schwann cells in PNS and may confirm the clinical assessment of neurodegenerative disorders such as multiple sclerosis and stroke (Jauch et al., 2006). Myelin Associated Glycoprotein (MAG) is selectively localized in periaxonal Schwann cell and oligodendroglial membranes of myelin sheaths, suggesting that it functions in glia-axon interactions in both the PNS and CNS (Schachner and Bartsch, 2000). Glial fibrillary acidic protein (GFAP) is expressed almost exclusively in astrocytes, where it is induced by neural injury and released upon disintegration of the astrocyte cytoskeleton (Rempe and Nedergaard, 2010). GFAP plays an essential role in maintaining shape and motility of astrocytic processes and contribute to white matter architecture, myelination and blood brain barrier (BBB) integrity (O'Callaghan et al., 2015). After traumatic brain injury (TBI), GFAP's serum concentration peaks at 2-6 h and has a half-life of <2 days (Diaz-Arrastia et al., 2014). S-100B exerts both detrimental and neurotrophic effects, depending on its concentration in brain tissues (Adami et al., 2001). After release, S-100B acts as a trophic factor for serotoninergic neurons, and plays a role in axonal growth and synaptogenesis during development. Thus, traumatic acute injury results in great destruction of astrocytes leading to massive release (50 to 100 fold) of S-100B into plasma, whereas S-100B levels in psychiatric disorders were only about 3 times higher in patients compared to controls (Uda et al., 1998; Arolt et al., 2003), correlating well with its neuroprotective action. Specifically, S-100B stabilizes tau and MAP-2. Its half-life in the serum is 2 h (Zurek and Fedora, 2012).

A recent study of airline pilots and other flight crewmembers chronically exposed to organophosphates through combustion of engine oil and hydraulic fluid that contain organophosphate esters resulted in symptoms similar to those reported by GW veterans (fatigue, headaches, confusion and memory problems). Interestingly, these crew members showed significantly elevated numbers of autoantibodies in their blood serum of CNS damage markers including those associated with axonal transport (microtubule associated protein-2 (MAP-2), tubulin, neurofilament triplet proteins (NFP) and microtubule associated protein-tau (tau protein)) and those exclusively associated with CNS glial activation and neuroinflammation (myelin basic protein (MBP), and glial fibrillary acidic protein (GFAP) (Abou-Donia et al., 2013). A follow-up histopathology autopsy study was performed on a deceased pilot with organophosphate exposure that confirmed CNS damage and demyelination (Abou-Donia et al., 2014). Specifically, the histopathology results showed axonal degeneration and demyelination and the post-mortem and pathological examination of the nervous system confirmed the autoantibody biomarker results.

Recent studies with GW veterans have shown persistent signs and symptoms characteristic of CNS injury including brain imaging and cognitive studies (White et al., 2016; Chao et al., 2010, 2011, 2014, 2016; Heaton et al., 2007; Sullivan et al., 2003). There are, however, no validated objective diagnostic tests to identify acute or chronic sequelae of brain injury in this veteran group. Diagnosis of brain injury using cranial computed tomography (CT) scan and magnetic resonance imaging (MRI) techniques such as diffusion tensor imaging (DTI), have not been able to clinically diagnose veterans with GWI because there have been no proven cutoff values for volumetric or other imaging parameters that have been able to provide the required near 100% accuracy in terms of sensitivity/specificity at the individual level to distinguish cases from controls needed for a diagnostic test. Imaging studies have been able to show differences and altered CNS functioning between veterans with GWI and healthy controls but have not yet been able to identify the groups diagnostically because of the significant overlap between the groups (Chao et al., 2010, 2011, 2014, 2016; Heaton et al., 2007). Hence, it is important to develop clinically available, simple and inexpensive biomarkers for detection of neuronal and glial injury essential in the diagnosis and understanding of the temporal progression of CNS damage in GWI. Recently, serum biomarkers such as cytoskeletal proteins, resulting from axonal degeneration, have been used in diagnosing brain injury (particularly traumatic brain injury). The use of these biomarkers is usually measured in serum shortly after brain injury, because they have short half-lives (Zurek and Fedora, 2011; Diaz-Arrastia et al., 2014).

However, many years have elapsed since the time that GW veterans returned from deployment and became ill therefore, this particular approach cannot apply to GWI. Based on results from both chronic and acute injury, we used our novel autoantibody biomarker panel described above for brain injury to test for the indication of CNS damage in veterans with chronic GWI (Abou-Donia et al., 2013, 2014). One prior study compared autoantibodies of myelin basic protein (MBP) and striated muscle antibodies in GW veterans and reported higher MBP and muscle antibodies in veterans with GWI (Vojdani and Thrasher, 2004). Autoantibodies have previously been recognized as potential objective biomarkers of GWI (Golomb, 2012). Therefore, we hypothesized that chemical exposure to pesticides, anti-nerve gas pills and/or sarin nerve gas during deployment in veterans with GWI caused an excitotoxic cascade (through potential glutamatergic, oxidative stress and proinflammatory cytokine signaling) resulting in neurodegeneration and apoptotic loss of brain cells, leading to blood brain barrier leakage of specific neuronal and glial proteins into circulation, with subsequent formation of autoantibodies (AB) against these proteins (Abou-Donia et al., 2013; Banks and Lein, 2012; Golomb, 2008; Terry, 2012; Binukumar and Gill, 2010; Soltaninejad and Abdollahi, 2009). In this study, we determined circulating IgG-class autoantibodies in serum from 20 GWI cases and 10 symptomatic (low back pain) controls against the following 9 brain proteins: neurofilament triplet proteins (NFP), tubulin, microtubule associated protein-tau (tau proteins), microtubule associated protein-2 (MAP-2), calcium/calmodulin Kinase II (CaMKII), myelin basic protein (MBP), myelin associated glycoprotein (MAG), glial fibrillary acidic protein (GFAP) and S-100B.

### Materials and Methods

### Materials

The sources of proteins were: NFP (bovine spinal cord), tau protein (human), MAP-2 (bovine serum), tubulin (bovine brain), and MBP (human brain), from Sigma-Aldrich (Saint Louis, Mo.); CaMKII (Human) recombinant Protein and MAG recombinant Protein from Novus Biologicals, Littleton, Colo., GFAP (human) from Biotrend Chemikalien GmbH, (Cologne, Germany) and S-100B (human brain) from American Qualex International, Inc. (San Clemente, Calif.). Horseradish peroxidase-conjugated goat anti-human IgG, and enhanced chemiluminescence reagent were obtained from Amersham Pharmacia Biotech (Piscataway, N.J.). SDS gels, 2-20% gradient (8×8), and tris-glycine 15 mM were obtained from Invitrogen (Carlsbad, Calif.). All other materials were purchased from Amersham.

### Ethics Statement

Approval for the use of stored blood samples for this study was obtained from the Duke University Medical Center Institutional Review Board.

### Case and Control Samples

Serum samples from 20 GWI cases with GWI and 10 non-veteran symptomatic controls with lower back pain were tested in this pilot study. GW veteran serum samples were collected from a study of acupuncture treatment in veterans with GWI from 2010 to 2012 (Conboy et al., 2012). Control serum samples were derived from a separate study of non-veteran patients with chronic lower back pain who served as ‘symptomatic low back pain’ controls from 2011 to 2013 (Jacobson et al., 2015). Veterans with GWI will be referred to as ‘cases’ and low-back pain symptomatic controls will be referred to as ‘controls’.

### Description of the Patient Cohorts:

### GWI-Case Cohort

“The Effectiveness of Acupuncture in the Treatment of Gulf War Illness” PI: Conboy, (Aug. 21, 2010-Dec. 26, 2012) N=104; Study Site: New England School of Acupuncture (NESA). Cases were recruited through the Defense Manpower Data Base (DMDC) personnel listings and advertisements. Cases were screened for GWI symptoms and were required to meet the CDC diagnostic criteria for chronic multi-symptom illness (CMI) in order for inclusion in the parent study and in the current study (Conboy et al., 2012; Fukuda et al., 1998). Inclusion in the current study also required that veterans were deployed to the 1990-1991 Gulf War. CMI is characterized by one or more symptoms of at least 6 months duration from at least two of three symptom categories: 1) fatigue; 2) mood-cognition; 3) musculoskeletal pain.

Symptoms were not necessarily required to have started during or after the Gulf War deployment. Exclusionary criteria included that the veteran was 1) currently enrolled in another clinical trial 2) Had another disease that likely could account for the symptoms, as determined by the Medical Monitor 3) Severe psychiatric illness (in the last 2 years psychiatric hospitalization, suicidal attempt, alcohol or substance abuse, use of antipsychotic medication) 4) Unable to complete the protocol based on the evaluation of the Medical Monitor.

cLBP-Cohort

“Structural Integration for chronic low back pain” PI: Jacobson (Mar. 4, 2011-Jun. 21, 2013) N=46. Study Site: Spaulding Rehabilitation Hospital (SRH). In this cohort, 46 outpatients from the Boston area with chronic nonspecific low back pain were randomized to parallel 20-week long treatment groups of structural integration (SI) plus outpatient rehabilitation (OR) versus OR alone. The details of the study are described in a recent publication (Jacobson et al., 2015). Inclusion criteria for the parent study included: (i) Men and women aged 18-65, (ii) cLBP of ≥6 months duration, not attributed to infection, neoplasm, severe radiculopathy (as indicated by frequent severe pain radiating down a leg), fracture, or inflammatory rheumatic process, (iii) bothersomeness of back pain self-rated on average over the preceding 6 months ≥3 on an 11-point ordinal scale (0=none, 10=worst imaginable), (iv) prior arrangement to enter a course of outpatient physical therapy for low back pain at a Boston area rehabilitation clinic, (v) English language fluency and mental capacity sufficient to provide informed consent and participate in the study. Exclusion Criteria for the study included: (i) Impaired hearing, speech, vision, and mobility sufficient to interfere with participation in the study, (ii) current or anticipated receipt of payments from Worker's Compensation or other insurance for disability attributed to low back pain, (iii) prior treatment with any SI therapy, (iv) plans to initiate additional treatment for back pain during the period of the study other than outpatient rehabilitation care, particularly massage or other manual therapies (e.g., chiropractic or osteopathic manipulation), (v) exclusions for safety: unresolved musculoskeletal pathology of the lower limbs, current pregnancy, any implanted medical device, osteoporosis, any hypercoagulation condition, eczema, skin infection, deep vein thrombosis, burns or other acute trauma including unhealed bone fractures or open wounds, psoriasis, psychiatric illness not well controlled, or current episode of exacerbated major depressive disorder.

### Collection and Storage of Samples

Samples from the GWI-cohort and the cLBP-cohort were all collected from the Boston area at the same time period at two different sites from 2010 to 2013. All sites followed exactly the same protocol for venipuncture, blood handling, serum separation, aliquoting and storage at −80° C. The same phlebotomy and sample protocol was distributed in writing to all sites. All samples analyzed were baseline blood samples collected pre-intervention therapy. Samples used for this study have not been previously thawed and are free of hemolysis by visual inspection (Tuck et al., 2009).

### Participant Demographics

The participant demographics indicate that a total of 20 veterans with GWI, 18 males and 2 females, compared to 6 females out of 10 cLBP controls participated in the study. The age of the GWI cases ranged from 38 to 61 (mean±SD 46.0±6.8) compared to 25 to 64 (mean±SD 50±11.4) years for controls; all study participants were white (Table 1). Seventy percent of veterans with GWI reported taking PB pills during the war (n=14). The groups differed with respect to gender (X2=8.5; p b 0.05) with significantly more women in the control group but did not differ with respect to age (t-value=−1.3; p N 0.05).

TABLE 1
Study Participant Demographics^{a}

| Demographics | Cases | Controls |
|---|---|---|
| Age (mean ± SE) | 46 (6.4) | 50 (11.4) |
| Gender (% female)* | 10 | 60 |
| Race (% Caucasian) | 100 | 100 |

Age range of Cases = 38-61 years and Controls = 25-64 years in 2010-2013 when the blood was collected
^{a}A total of 20 subjects and 10 controls participated in the study.
*Cases were significantly different from controls for gender p < 0.05 but not for age.

### Western Blot Assay

To screen for the presence of autoantibodies against a battery of proteins, we applied a Western blot approach as previously reported (Abou-Donia et al., 2013). Each serum sample was analyzed in triplicate. Each protein was loaded as 10 ng/lane except for IgG that was loaded as 100 ng/lane. Proteins were denatured and electrophoresed in SDS-PAGE (4% to 20% gradient) purchased from Invitrogen (Carlsbad, Calif.). One gel was used for each serum sample. The proteins were transferred into polyvinylidene fluoride (PVDF) membranes (Amersham Pharmacia Biotech Piscataway, N.J.). Nonspecific binding sites were blocked with Tris-buffered Saline-Tween (TBST) (40 mM Tris [pH 7.6], 300 mM NaCl, and 0.1% Tween 20) containing 5% non-fat drymilk for 1 h at 22° C. Membranes were incubated with serum samples at 1:100 dilutions in TBST with 3% non-fat dry milk overnight at 4° C. After five washes in TBST, the membranes were incubated in a 1:2000 dilution of horseradish peroxidase-conjugated goat anti-human IgG (Amersham Pharmacia Biotech (Piscataway, N.J.). The dot blots were probed with anti-human IgG (H+L) HRP conjugate antibody (Cat. No. 31410, Thermo Fisher Scientific Inc., Pittsburgh, Pa., USA) for 1 h at RT, incubated with ECL reagent (Cat. No. 34096). The membranes were developed by enhanced chemiluminescence using the manufacturer's (Amersham Pharmacia Biotech) protocol and a Typhoon 8600 variable mode imager. The signal intensity was quantified using Bio-Rad image analysis software (Hercules, Calif.). All tests were performed with the investigators blinded to participant diagnosis.

### Specificity of Sera Autoantibodies

Previously we checked the specificity of the serum autoantibody by performing peptide/antigen competition assay, in which the serum was spiked with the target protein or peptide (Abou-Donia et al., 2013). The serum from random healthy controls was mixed with or without tau, MAP or MBP. The serum/protein mix was centrifuged at 15,000 rpm to pellet any immune complexes. The supernatants were then carefully removed and used in Western blotting.

### Calculations

The mean value of the optical density measurement from the triplicate testing was used for each serum sample tested and normalized by total IgG. Thus, the results are expressed as mean values of triplicate assays of optical density arbitrary units normalized to total serum IgG.

### Power Analysis

A total of 20 GWI cases were available for testing in this convenience sample. Effect size calculations were based on two-sample t-test assuming a common standard deviation between groups. The power analysis assumes that cases and controls are not matched. In a t-test of difference between two independent means, selecting power of 80%, 2-sided alpha 0.05, and size of 20 vs 10, the study was powered to detect an effect only if at least 1.12 SD.

### Statistics

Grouped data are reported as mean±SD. The values from cases were compared to the control group using t-tests and Pearson correlation analyses (SigmaStat, Systat Software) and p-values were calculated. Pairwise correlations among the nine biomarkers were assessed. A 2-sided p value <0.05 was considered significant. Due to the exploratory nature of this pilot study, analyses were not adjusted for multiple comparisons.

### Results

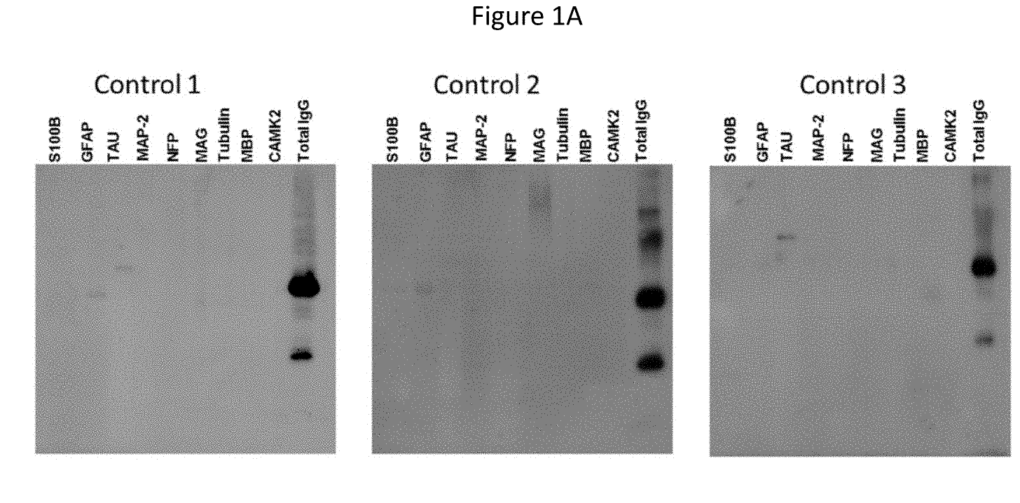

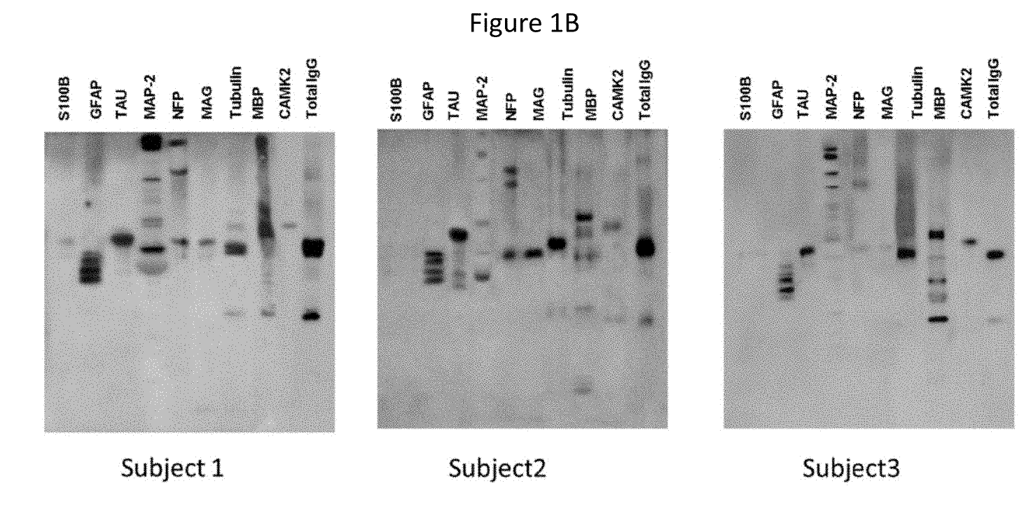

As previously described, we assessed the specificity of the serum autoantibody by performing peptide/antigen competition assay, in which the serum was spiked with the target protein or peptide. The serum bound to tau eliminated the tau band in the Western blot (see FIG. 1) while the band of MAP-2 or MBP were present and not affected. The serum bound to MAP-2 eliminated the MAP-2 band in the Western blot while the band of tau or MBP was present. The serum bound to MBP eliminated the MBP band in the Western blot while the bands of tau and MAP-2 were present. These results indicate that each autoantibody in the serum was specifically neutralized by its target protein in serum sample and was no longer available to bind to the epitope present in the protein on the Western blot. This confirmed that the assay used in this study, was specific and accurately determined autoantibodies against tested proteins in serum samples.

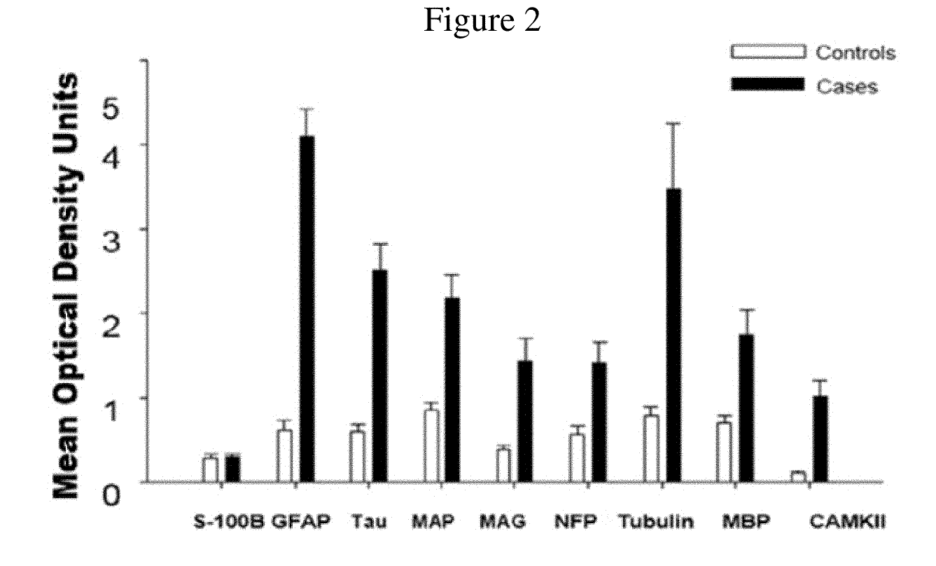

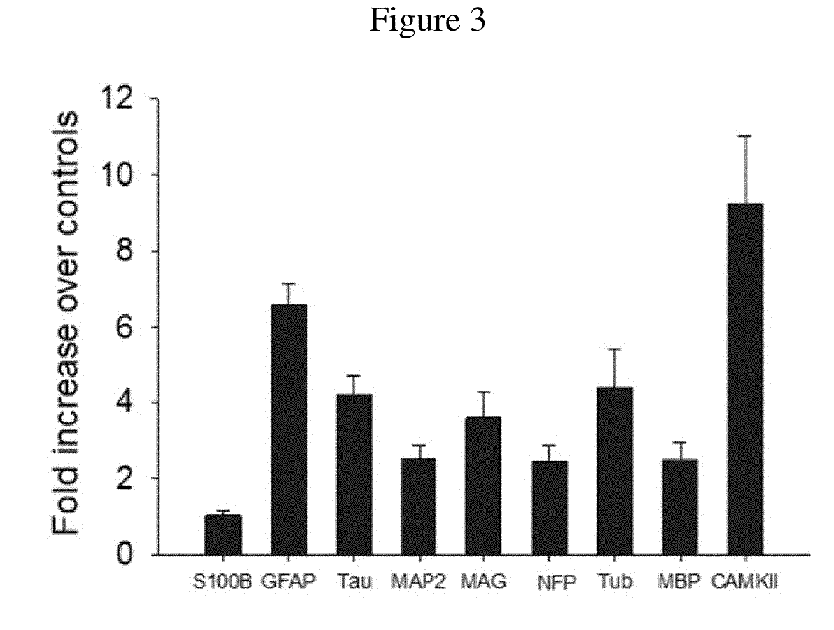

To detect autoantibodies in serum, we probed Western blots with individual serum samples. A total of 30 human serum samples (20 veterans with GWI and 10 non-veteran symptomatic low-back-pain controls) underwent measurement of the levels of the serum circulating IgG-class autoantibodies against nine neuronal- and glial-specific proteins. Table 2 lists the number of GWI cases who were exposed to chemical and environmental exposures. It shows that 14 cases (70%) used PB as a prophylaxis against possible exposure to nerve agents and nine cases reported being exposed to the nerve agent sarin. In addition, a total of eight cases reported receiving notification from the Department of Defense (DOD) that they were potentially exposed to sarin and other chemicals due to their proximity to the Khamisiyah, Iraq underground weapons depot where a chemical weapons cache was destroyed in March 1991 (US DOD, 2002). Eight cases reported exposure to depleted uranium. All of the cases reported exposure to one or more insecticides or a mixture of pesticides including organophosphates, carbamates, pyrethroids and organochlorines. Eleven cases used the insect repellant DEET. All cases underwent environmental and other exposures listed in Table 2. Other chemicals that the cases reported exposure to included oil well fires, sand, tent heaters, jet fuel, and solvents. Some veterans reported exposure to malaria and 18 reported being vaccinated. Serum from GWI cases showed significantly increased levels of autoantibodies against all cytoskeletal proteins except those against S-100B compared to non-veteran symptomatic (low back pain) controls (Table 3). Due to the gender differences between the cases and controls, analyses were also run with just the males in the groups. Although there was only a small number of males (n=4) in the control group which could be problematic in this type of analysis, results of this comparison showed a very similar pattern of significant differences in all autoantibodies (GFAP p<0.001; Tau p<0.001; MAP p<0.002; MAG p<0.001; PNF p<0.006; Tubulin p<0.003; MBP p<0.01; S-100B p=0.31). The majority of GWI serum reacted intensely to neural proteins, while most control serum showed a weak or no reaction. FIGS. 1A and 1B present Western blot results from three representative GWI cases and three controls. The levels of serum autoantibodies in GWI cases and controls to neural-specific proteins expressed as mean values±SD of triplicate assays of optical density arbitrary units normalized to total serum IgG optical density ranged from 0.30 for S-100B and 4.09 for GFAP for the cases compared to 0.30 and 0.62, respectively for controls are listed in Table 3 and shown in FIG. 2. The percentage of autoantibodies against neural proteins of cases compared to controls (in descending order) were: CaMKII, 927, GFAP 660, Tau 483, Tubulin 441, MAG 360, MBP 250, NFP 245, MAP-2 230, S-100B 103. FIG. 3 presents the mean values ±SD (p b 0.001) of fold increase of autoantibodies against neural proteins for the cases compared with the controls. Serum from controls had no or low levels of circulating autoantibodies to nervous system-specific biomarkers. Autoantibodies against CaMKII were more predominant in the cases' serum than in controls' serum (FIG. 3).

TABLE 2
Chemicals, Environmental and other exposures
of Subjects during the Gulf War ^{a}

| | Chemical | | | Environmental and | |
|---|---|---|---|---|---|
| | Exposures | | | other exposures | |
| | Exposed | % | | Exposed | % |
| Pyrdostigmine | 14 | 70 | Deployed in | 8 | 40 |
| Bromide (PB) | | | Khamisiyah | | |
| Organo- | 7 | 35 | Contaminated | 18 | 90 |
| phosphorus | | | Food/Water | | |
| Pesticides | | | | | |
| (OP) | | | | | |
| Carbamates | 7 | 35 | Vaccines | 18 | 90 |
| Pyrethroids | 4 | 20 | Malaria | 12 | 60 |
| DEET | 11 | 55 | Sand | 18 | 90 |
| Sarin | 9 | 45 | Tent Heater | 11 | 55 |
| Depleted | 6 | 30 | Jet Fuel | 14 | 70 |
| Uranium | | | | | |
| (DU) | | | | | |
| Solvents | 10 | 50 | Oil Fires | 18 | 90 |

^{a} A total of 20 veterans with GWI participated in the study

TABLE 3
Unpaired Statistical analysis of the Levels ^{a} of Serum Autoantibodies (AA)
in Symptomatic Controls ^{b} and GWI Cases ^{b} to Neural-Specific Proteins ^{c}

| | NFP | Tau | Tubulin | MBP | MAG | MAP2 | GFAP | S100B | CaMKII |
|---|---|---|---|---|---|---|---|---|---|
| Cases ± | 1.42 ± 0.24 | 2.52 ± 0.31 | 3.48 ± 0.78 | 1.75 ± 0.30 | 1.44 ± 0.28 | 2.18 ± 0.29 | 4.09 ± 0.33 | 0.30 ± 0.03 | 1.02 ± 0.20 |
| SE | | | | | | | | | |
| Controls ± | 0.58 ± 0.09 | 0.60 ± 0.09 | 0.79 ± 0.11 | 0.70 ± 0.11 | 0.40 ± 0.04 | 0.086 ± 0.09 | 0.62 ± 0.11 | 0.29 ± 0.04 | 0.11 ± 0.03 |
| SE | | | | | | | | | |
| p Values | 0.02 | 0.0001 | 0.001 | 0.001 | 0.007 | 0.002 | 0.00001 | 0.4020 | 0.015 |

^{a} The results are expressed as mean values of triplicate assays of optical density arbitrary units normalized to IgG optical density as fold of healthy controls.
^{b} Values from cases were compared to the control group using t-tests; most were highly significant p < 0.001 (2-sided), except for S-100B that was not significantly different from controls. Cases were significantly different from controls with respect to gender p < 0.05 but not with respect to age.

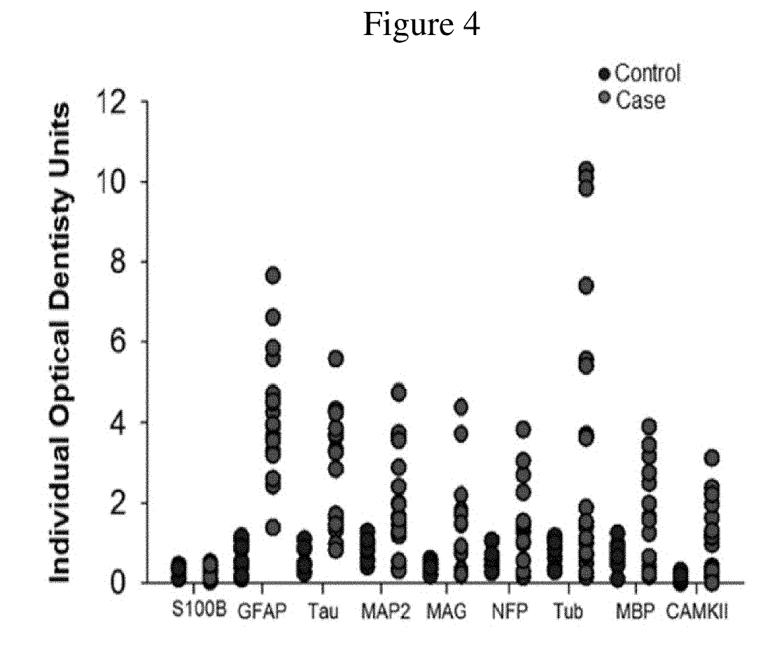

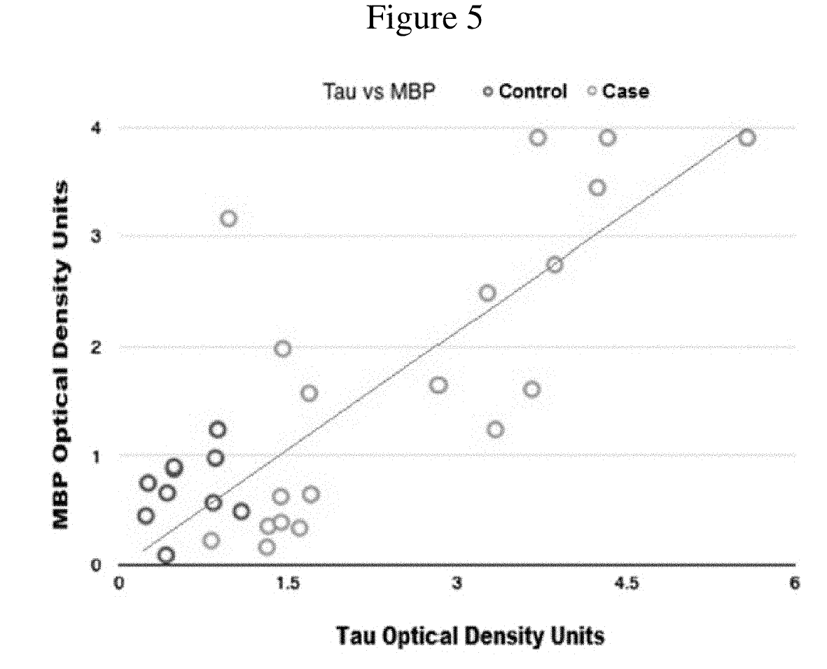

FIG. 4 shows that Tubulin and GFAP had the highest values in the GWI cases compared with the controls. Pairwise correlations among the nine autoimmune biomarkers were significant only for the pair Tau and MBP. When comparing the correlation between each pair, only tau and MBP were significantly linearly correlated to each other (FIG. 5). FIG. 5 shows that the control values of those two biomarkers were <1 optical density unit, whereas GWI cases had values strongly linearly correlated with each other such that on average tau was elevated up to 10 times higher than controls in some GWI cases, and MBP was also elevated up to 5 times higher for the same cases vs the controls.

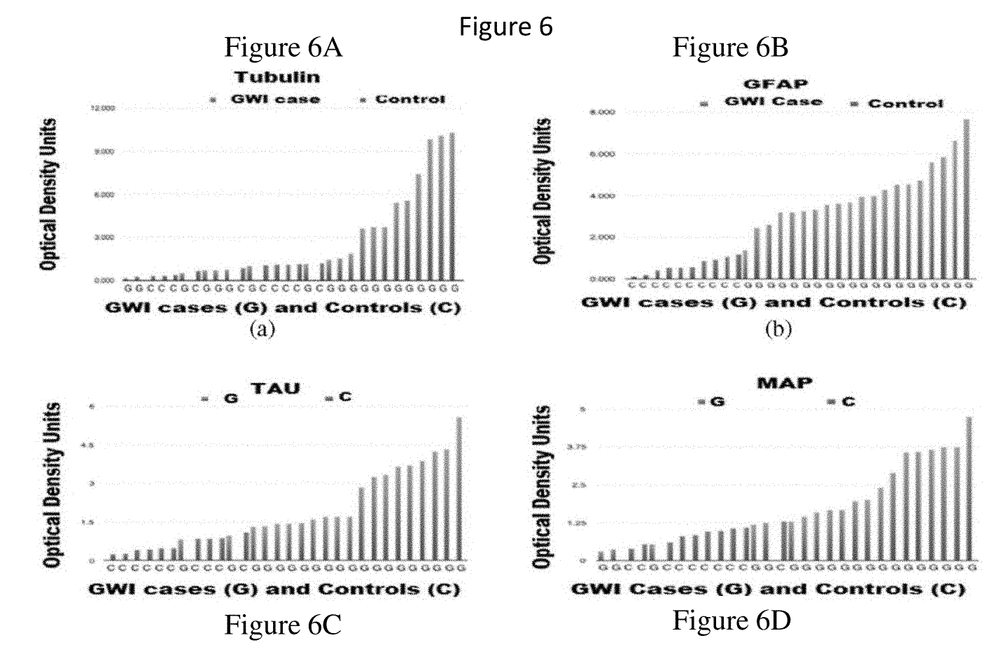

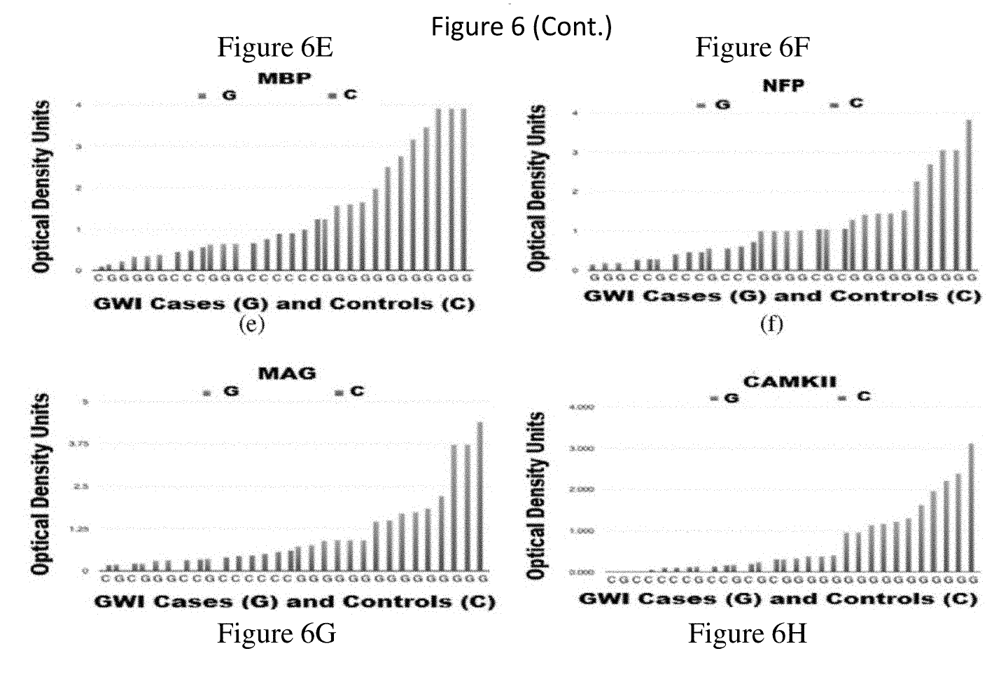

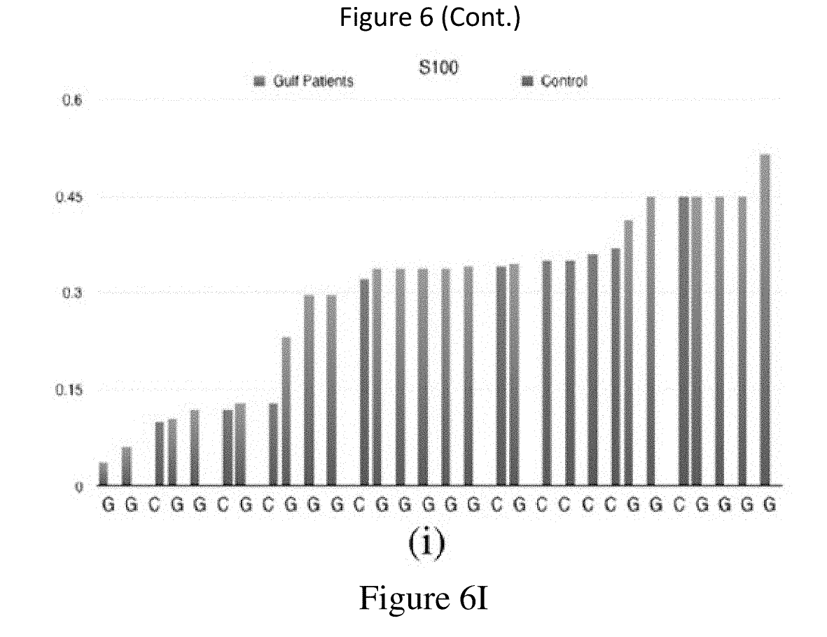

Finally, when each biomarker was compared separately between individual cases and controls for potential fold-increase cut-points to discriminate the groups, results indicated that tubulin values had some of the highest-fold increased values in the individual GWI cases compared with the individual control values although only 60% of the individual cases (n=12) showed that effect (FIG. 6A). However, in 9 (out of the 20) cases tubulin values were elevated by a factor of 3 to 9-fold higher than the controls. In FIG. 6B, GFAP was elevated the most in cases compared to controls. In fact, GFAP was higher in all of the cases compared with all of the controls with 20 out of 20 cases having 2 to 7 fold higher value than the control mean. Thus GFAP values completely distinguished the cases from the controls. GFAP values did not overlap in cases vs controls in this small sample; however, the separation in the ranges was small relative to the substantial standard deviations. In FIG. 6C, tau was higher than controls in 18 cases and 50% of the cases had double the value of tau compared with the controls. In FIG. 6D, MAP was higher than the controls in 15 cases and 75% of the cases had a 0.5 to 11-fold higher value than the controls. In FIG. 6G MAG was higher than controls in 15 cases and 75% of the cases had up to a 10-fold higher value than the controls. In FIG. 6F NFP was higher than controls in only 50% of the cases (n=10) and they showed 0.5 to 11-fold higher values than controls. In FIG. 6E, MBP was higher than controls in 12 cases and 60% of the cases were higher than controls with 2 to 5-fold higher values than controls. In FIG. 6H, CAMKII was higher than controls in 16 cases and 50% of the cases had a 3 to 30-fold higher value than the controls. S100B values, as shown in FIG. 6I, were not statistically significant as the values overlapped with cases and controls.

### Discussion

This pilot study reports significantly elevated levels of autoantibodies against neurotypic- and gliotypic-specific proteins in serum from a sample of 20 veterans with GWI and 10 non-veteran symptomatic (low back pain) controls with musculoskeletal symptoms rather than CNS symptoms. The increased levels in GWI cases compared to controls ranged from 9.27 fold for CaMKII to 6.6 fold for GFAP to 2.45 fold for neurofilaments. Autoantibody levels against S-100B were not different in GWI cases than controls (1.03 fold) consistent with its neural protective role and in agreement with presence of chronic injury and absence of acute brain injury in veterans with GWI (Zurek and Fedora, 2011; Diaz-Arrastia et al., 2014; Stalnacke et al., 2006, 2004; Coch and Leube, 2016). Previous studies, using animal models of GWI, showed that exposure to the neurotoxicants that were present in the GW environment, caused deficits in behavioral outcomes that were accompanied by neuronal and glial degeneration (Abdel-Rahman et al., 2001, 2002a,b, 2004a,b; Abou-Donia et al., 2000, 2001, 2004). Following neurodegeneration, there is accumulation of cellular neurological waste products or debris such as misfolded or hyper-phosphorylated proteins that form toxic stable aggregates (Nedergaard, 2013; Edgar et al., 2004). This extracellular debris sends damage signals that cause the CNS immune cells—microglia—to become activated and act as profound antigen presenting cells that secrete pro-inflammatory cytokines (IL-1β, TNF-α and IL-6) and mediators (reactive oxygen species, ROS) resulting in the recruitment of T-lymphocytes (Milligan and Watkins, 2009; Banks and Lein, 2012). Multiple exposures to these waste proteins can cause microglia and astrocytes to become primed to react more strongly after each subsequent exposure (Watkins and Maier, 2003). This can result in a persistent neuroimmune response and chronic neuroinflammation contributing to chronic health symptoms, such as those seen in GW veterans (Johnson et al., 2016; Milligan and Watkins, 2009; Maier and Watkins, 1998; Watkins and Maier, 2003). These waste proteins are eventually released into circulation due to defects in the brain-blood barrier induced by astrocyte alterations. Waste proteins in the brain ultimately reach the liver through a mechanism known as the “glymphatic system” where they are degraded (Nedergaard, 2013). However, the released proteins that could serve as markers of injury are present in the short-term and cannot be used as biomarkers in the case of chronic GWI (Zurek and Fedora, 2011; Diaz-Arrastia et al., 2014). Thus detection of autoantibodies can serve as surrogate markers for these circulating waste proteins as described in this study.

The highest increase in autoantibodies was against CaMKII which was 9.27 times higher than that of controls followed by GFAP which was 6 times higher than controls. This result is consistent with the veterans' exposure during their deployment to the Gulf War to organophosphorus compounds such as pesticides, and the nerve agent sarin that have been shown to increase the activity and mRNA expression of CaMKII (Patton et al., 1983, 1985, 1986; Gupta et al., 1998; Barbier et al., 2009) as well as enhanced CaMKII-induced phosphorylation of NFP, tubulin (Serrano et al., 1986) and tau activity leading to the aggregation, deregulation and accumulation of NFP (Abou-Donia et al., 1993; Norgren et al., 2003) and tubulin in the axon (Abou-Donia, 1993; Jensen et al., 1992, Gupta et al., 2000; Grigoryan and Lockridge, 2009). Aggregated neurofilaments result in slowing of axonal transport as has been illustrated in GW-relevant animal and cell neurotoxicant models (Gupta et al., 1997; Reagan et al., 1994; Terry et al., 2012; Gao et al., 2016; Edgar et al., 2004). GW-relevant exposure models have also been associated with astrocyte activation (Zakirova et al., 2015; Ojo et al., 2014).

Neuronal proteins studied in this pilot analysis represented various anatomical regions of the neuron with distinct functions which can be instructive with regard to the pathobiology of GWI (Lapadula and Abou-Donia, 1992). All of the proteins used are involved in axonal structure and function and are released as products of neural degeneration of various regions of the neuron. MAP-2 is present in the dendrites; CaMKII, tau, tubulin, and neurofilament proteins are located in the axon; myelin basic protein (MBP) and myelin associated glycoprotein (MAG) are an integral part of myelin (McMurray, 2000). Furthermore, the central nervous system-specific glial protein, GFAP and S-100B are secreted by astrocytes after neuronal injury (McMurray, 2000). Following axonal and myelin degeneration, neuronal and glial proteins are released and once in circulation, activated lymphocytes, B and T cells lead to the formation of autoantibodies against these proteins (Schwartz and Shechter, 2010a,b).

Increased autoantibodies against nervous system-specific proteins leads to structural consequences in various regions as follows: increased autoantibodies against neurofilaments proteins, tau, CaMKII and tubulin are indicative of axonal degeneration; increased autoantibodies against MAG and/or MBP suggest demyelination, increased autoantibodies against MAP-2 suggest dendritic degeneration, increased autoantibodies against GFAP suggest astrogliosis, and the low or no-increased levels of autoantibodies against S-100B is consistent with chemical-induced brain injury (Zurek and Fedora, 2011, Diaz-Arrastia et al., 2014; Stalnacke et al., 2006, 2004). The linear correlation pattern of tau and MBP in this study suggests an important potential effect of axonal degeneration followed by demyelination that would correspond with prior neuroimaging studies in neurotoxicant exposed GW veterans (Heaton et al., 2007; Chao et al., 2010). Furthermore, these structural changes of the nervous system lead to functional alterations. Hence axonal degeneration in the cerebral cortex leads to motor and sensory abnormalities, ataxia, deficit in posture, locomotion, and skilled fine motor movements (fingers, speech, facial expression) and weakness; degeneration of the limbic system including the hippocampus leads to: learning and memory deficits, and neurobehavioral (mood, emotion and judgment) abnormalities; increased autoantibodies against MAP-2 suggests damage to the dendrite-rich Purkinje cells in the cerebellum resulting in: gait and coordination abnormalities, staggering gate and ataxia (McMurray, 2000; Abou-Donia, 2015). Increased autoantibodies against GFAP indicate astrogliosis and potential neuroinflammation and/or glial scarring. GFAP contributes to white matter architecture, myelination and blood brain barrier (BBB) integrity (O'Callaghan and Sriram, 2005; Amourette et al., 2009; Lamproglou et al., 2009). Consequently, blood levels of GFAP in healthy individuals are very low. GFAP levels were higher in GWI cases and completely discriminated between the cases and controls in this study. This is particularly relevant because disorders with higher levels of GFAP include memory disorders such as Alzheimer's and vascular dementia that have significant axonal neurodegeneration and neuroinflammation (Mecocci et al., 1995). Increased autoantibodies against 5-100B suggest traumatic brain damage and can help to differentiate between acute and chronic brain injury (Stroick et al., 2006; Stalnacke et al., 2006, 2004; Zurek and Fedora, 2011; Diaz-Arrastia et al., 2014; Coch and Leube, 2016). Their lack of increase in this study suggests against acute traumatic brain injury in veterans with GWI.

Important mechanistic clues from animal and cell studies of these GW-relevant neurotoxicants have shown deficits in axonal transport, as well as aberrations in neurofilaments and microtubules, which are the structural railways for axonal transport (Gupta and Abou-Donia, 1995a, b; Gearhart et al., 2007; Grigoryan and Lockridge, 2009; Prendergast et al., 2007, Jiang et al., 2010). Mitochondria are also delivered by axonal transport to provide the energy required to power the biochemical reactions necessary for the functioning of the axon and have shown altered functioning in GW-relevant neurotoxicant models (Middlemore-Risher et al., 2011). GW-relevant chronic low-level organophosphate exposure has also been associated with mitochondrial compromise from oxidative stress induction and with neuroinflammation resulting in cell damage or cell death resulting in debris of waste proteins in the extracellular spaces (Laetz et al., 2009; Kaur et al., 2007; Banks and Lein, 2012). In fact, one hypothesis of GWI suggests that mitochondrial damage and oxidative stress in the brain and the periphery have caused the chronic symptoms of GWI; notably, increased autoantibodies were expressly cited among objective markers and mediators in this model (Golomb et al., 2014; Golomb, 2012; Koslik et al., 2014).

Another hypothesis of GWI suggests that the neurotoxicants acted synergistically to create a self-perpetuating neuroinflammatory state, which in turn has an ongoing negative impact on brain cells including neurons (microtubules, motor proteins, mitochondria) and glia (microglia, astrocytes, oligodendrocytes) and blood-brain barrier function (O'Callaghan and Sriram, 2005). Clinical studies have also found consistent results with GW veteran cohorts who showed impaired cognitive functioning and reduced volume and altered white matter microstructural integrity on MRI in OP pesticide, sarin nerve agent and PB pill exposed cohorts (White et al., 2016; Sullivan et al., 2013; Chao et al., 2010; Heaton et al., 2007; Proctor et al., 2006; Sullivan et al., 2003). These prior results suggest clear CNS alterations in neurotoxicant exposed GW veterans which correlated with behavioral outcomes that are related to neurodegeneration and perhaps with both a chronic neuroinflammatory and Mitochondrial/OS hypothesis.

The only other study that we are aware of that compared CNS autoantibodies in GW veterans compared MBP and striated and smooth muscle antibodies and reported higher MBP and muscle antibodies in veterans with GWI when compared with controls (Vojdani and Thrasher, 2004). The current study validates the prior MBP findings and expands on those findings with a larger panel of 8 additional CNS autoantibody markers. Collectively, these findings suggest that alterations in white matter as evidenced by circulating autoantibodies to MBP appear to be associated with GWI. This finding corresponds with both leading hypotheses for GWI given that white matter alterations can be associated with oxidative stress and neuroinflammation as a result of glial activation and signaling of both proinflammatory cytokines and oxidative stress (Milligan and Watkins, 2009). The additional finding of this study that higher Tau autoantibody levels were significantly linearly correlated with higher MBP autoantibody levels in GWI cases suggests that axonal degeneration may be occurring before demyelination in veterans with GWI and warrants a further more conclusive study to distinguish it from the more myelin-specific toxic leukoencephalopathies (Schmahmann et al., 2008; Filley, 2013). These findings also correspond with MRI findings of differences on both white and gray matter brain volumes in neurotoxicant-exposed GW veterans (Heaton et al., 2007; Chao et al., 2010, 2011, 2014, 2016). These findings also clearly suggest that glia and astrocytes in particular should be further studied in GWI given significantly higher levels of GFAP in the GWI cases that correspond with prior animal models of GWI (Abdel-Rahman et al., 2001, 2002a, 2002b, 2004a, 2004b; Abou-Donia et al., 2000, 2001, 2002, 2004; Zakirova et al., 2015; Ojo et al., 2014) and with recent studies illustrating the ability of astrocytes to donate mitochondria to damaged neurons (Hayakawa et al., 2016).

These results suggest a possible new avenue for further development of an objective biomarker of GWI. The strong results including 9-fold higher levels of CAMKII, 6-fold higher levels of GFAP and 4-fold higher levels of tau and tubulin that were presented in this study warrant further research for a blood-based objective marker of GWI in larger, well-characterized veteran cohorts. These results suggest a possible new avenue for further development of an objective biomarker of GWI. The identification of this small panel of neural-specific autoantibody biomarkers in GWI shows promise for further validation in larger study samples that are more carefully matched for subject demographics (particularly age), different types of control groups (i.e. healthy and CNS symptomatic groups) and that classify cases by both the CDC and the more specific Kansas GWI criteria which also specifies the time period of deployment which may be relevant to particular OP and other deployment-related exposures (Steele, 2000; Fukuda et al., 1998). Future directions will be to compare these CNS autoantibody markers with specific behavioral outcomes including cognitive performance and brain imaging of gray and white matter volume and microstructural integrity to further validate these suspected brain-immune-behavioral outcomes.

In conclusion, in this pilot study GWI was significantly associated with 2-9 fold increased serum autoantibodies against 8 neuronal and glial-specific proteins (CaMKII, GFAP, Tau, Tubulin, MAG, MBP, NFP, MAP-2) and not with a marker of more acute damage (S-100B). The autoantibodies that were found here to be elevated in GWI, targeted proteins/antigens that play critical roles in the structure and function of the neuron including axonal transport and myelination. Many of them are explicit markers for neurodegenerative disorders, consistent with axonal and myelin degeneration of myelinated neurons and with astrogliosis, cell signaling and neuroinflammation. These same proteins have been shown to be affected in other clinical groups and animal models with similar organophosphate and carbamate exposures (Abou-Donia et al., 2013, 2014). These results validate prior reports of increased MBP autoantibodies in GWI cases and suggest that oligodendrocyte signaling, glia and white matter alterations should continue to be further studied in GWI and validated with health symptom and behavioral outcomes (Vojdani and Thrasher, 2004). The results also indicate that veterans with GWI may be continuing to show brain neuronal degeneration and glial activation that would be consistent with recent reports of chronically persistent and in some cases worsening health of these veterans (Smith et al., 2013; Ozakinci et al., 2006; Li et al., 2011; Kang et al., 2009; Dursa et al., 2016; White et al., 2016).

### References for Example 1

Abdel-Rahman, A., Shetty, A. K., Abou-Donia, M. B., 2001. Exp. Neurol. 172, 153-171.
Abdel-Rahman, A. A., Shetty, A. K., Abou-Donia, M. B., 2002a Neurobiol. Dis. 10, 306-326.
Abdel-Rahman, A. A., Shetty, A. K., Abou-Donia, M. B., 2002b. Neuroscience 113, 721-741.
Abdel-Rahman, A. A., Goldstein, L. B., Bulman, S. L., Khan, W. A., El Masry, E. M., Abou-Donia,
M. B., 2004a. J. Toxicol. Environ. Health 67, 331-356.
Abdel-Rahman, Shetty, A. K., Abou-Donia, S. M., El-Masry, E. M., Abou-Donia, M. B., 2004b. J. Toxicol. Environ. Health 67, 163-192.
Abou-Donia, M. B., 1993. Chem. Biol. Interact. 87, 383-393.
Abou-Donia, M. B., 1995. Clin. Exp. Pharmacol. Physiol. 22, 358-359.
Abou-Donia, M. B., 2015. Neurotoxicity. In: Abou-Donia, Mohamed B. (Ed.), Mammalian Toxicology. John Wiley & Sons, UK, pp. 395-423.
Abou-Donia, M. B., Lapadula, D. M., 1990. Annu. Rev. Pharmacol. Toxicol. 30, 405-550.
Abou-Donia, M. B., Viana, M. E., Gupta, R. P., Knoth-Anderson, J., 1993. Neurochem. Int. 22, 165-173.
Abou-Donia, M. B., Wilmarth, K. R., Abdel-Rahman, A. A., Jensen, K. F., Oehme, F. W., Kurt, T. L., 1996a. Fundam. Appl. Toxicol. 34, 201-220.
Abou-Donia, M. B., Wilmarth, K. R., Jensen, K. F., Oehme, F. W., Kurt, T. L., 1996b. J. Toxicol. Environ. Health 48, 35-56.
Abou-Donia, M. B., Goldstien, L. B., Jones, K. H., Abdel-Rahman, A. A., Damodaran, T. B., Dechkovskaia, A. M., Bullman, S. L., Amir, B. E., Khan, W. A., 2000. Toxicol. Sci. 60, 305-314.
Abou-Donia, M. B., Goldstien, L. B., Dechovskaia, A., Bullman, S., Jones, K. G., Herrick, E. A., Abdel-Rahman, A. A., Khan, W. A., 2001. J. Toxicol. Environ. Health 62, 523-541.
Abou-Donia, M. B., Dechkovskaia, A. M., Goldstein, L. B., Bullman, S. L., Khan, W. A., 2002. Toxicol. Sci. 66, 148-158.
Abou-Donia, M. B., Dechkovskaia, A. M., Goldstein, L. B., Abdel-Rahman, A. A., Bulman, S. L., Khan, W. A., 2004. Pharmacol. Biochem. Behav. 77, 253-262.
Abou-Donia, M. B., Abou-Donia, M. B., El-Masry, E. M., Monoro, J., Mulder, M. F. A., 2013. J. Toxicol. Environ. Health 76, 363-380.
Abou-Donia, M. B., van de Goot, F. R. W., Mulder, M. F. A., 2014. J. Biol. Phys. Chem. 34-53.
Adami, C., Sorci, G., Blasi, E., Agneletti, A. L., Bistoni, F., Donato, R., 2001. Glia 33 (2), 131-142.
Amourette, C., Lamproglou, I., Barbier, L., Fauquette, W., Zoppe, A., Viret, R., Diserbo, M., 2009. Behav. Brain Res. 203 (2), 207-214.
Arolt, V., Peters, M., Erfurth, A., Wiesmann, M., Missler, U., Rudolf, S., Kirchner, H., Rothermundt, M., 2003. Eur. Neuropsychopharmacol. 13 (4), 235-239.
Ballough, G. P1., Martin, L. J., Cann, F. J., Graham, J. S., Smith, C. D., Kling, C. E., Forster, J. S., Phann, S., Filbert, M. G., 1995. J. Neurosci. Methods 61 (1-2), 23-32.
Banks, C. N., Lein, P. J., 2012. Neurotoxicology 33 (3):575-584. http://dx.doi.org/10.1016/j.neuro.2012.02.002.
Barbier, L., Diserbo, M., Lamproglou, I., Amourette, C., Peinnequin, A., Fauquette, W., 2009. Behav. Brain Res. 197 (2), 292-300.
Binukumar, B. K., Gill, K. D., 2010. Indian J. Exp. Biol. 48 (7), 697-709.
Chao, L. L., Rothlind, J. C., Cardenas, V. A., Meyerhof, D. J., Weiner, M. W., 2010. Neurotoxicology 31, 443-501 (2010).
Chao, L. L., Abadjian, L., Hlavin, J., Meyerhoff, D. J., Weiner, M. W., 2011. Neurotoxicology 32 (6):814-822. http://dx.doi.org/10.1016/j.neuro.2011.06.006 (December, Epub 2011 Jun. 29).
Chao, L. L., Kriger, S., Buckley, S., Ng, P., Mueller, S. G., 2014. Neurotoxicology 44:263-269. http://dx.doi.org/10.1016/j.neuro.2014.07.003 (September, Epub 2014 Jul. 21).
Chao, L. L., Reeb, R., Esparza, I. L., Abadjian, L. R., 2016. Neurotoxicology 53:246-256. http://dx.doi.org/10.1016/j.neuro.2016. 02.009 (March, Epub 2016 Feb. 23).
Coch, R. A., Leube, R. E., 2016. Cell 5 (3) (2016 Jul. 15).
Conboy, L., John, M. St, Schnyer, R., 2012. Contemp. Clin. Trials 33, 557-562.
Diaz-Arrastia, R1., Wang, K. K., Papa, L., Sorani, M. D., Yue, J. K., Puccio, A. M., P J, McMahon, Inoue, T., YuhEL, Lingsma H. F., Maas, A. I., Valadka, A. B., Okonkwo, D. O., Manley, G. T., 2014. J. Neurotrauma 31 (1), 19-25.
Directorate for Deployment Health Support of the Special Assistant to the Under Secretary of Defense (Personnel and Readiness) for Gulf War Illness Medical Readiness and Military Deployments, 2002. http://www.gulflink.osd.mil/khamisiyahiii (April, accessed Jul. 27, 2016).
Dursa, E. K., Barth, S. K., Schneiderman, A. I., Bossarte, R. M., 2016 January J. Occup. Environ. Med. 58 (1), 41-46.
Edgar, J. M., McLaughlin, M., Yool, D., Zhang, S. C., Fowler, J. H., Montague, P., Barrie, J. A., McCulloch, M. C., Duncan, I. D., Garbern, J., Nave, K. A., Griffiths, I. R., 2004. J. Cell Biol. 166 (1), 121-131 (July 5, Epub 2004 Jun. 28).
Erickson, B., He, Julie, Grumbach, Isabella M., Anderson, Mark E., 2011. Physiol. Rev. 91, 889-915 (2011).
Filley, C. M., 2013. Psychiatr. Clin. N. Am. 36 (2):293-302. http://dx.doi.org/10. 1016/j.psc.2013.02.008 (June, Epub 2013 Apr. 15. Review).
Fukuda, K., Nisenbaum, R., Stewart, G., Thompson, W. W., Robin, L., Washko, R. M., Noah, D. L., Barrett, D. H., Randall, B., Herwaldt, B. L., Mawle, A. C., Reeves, W. C., 1998. JAMA 280 (11), 981-988.
Gao, J., Naughton, S. X., Wulff, H., Singh, V., Beck, W. D., Magrane, J., Thomas, B., Kaidery, N. A., Hernandez, C. M., Terry Jr., A. V., 2016. J. Pharmacol. Exp. Ther. 356 (3):645-655. http://dx.doi.org/10.1124/jpet.115.230839 (March, Epub 2015 Dec. 30. PMID: 26718240).
Gearhart, D. A., et al., 2007. Toxicol. Appl. Pharmacol. 218 (1), 20-29.
Golomb, B. A., 2008. Acetylcholinesterase inhibitors and Gulf War illnesses. Proc. Natl. Acad. Sci. U. S. A 105 (11):4295-4300. http://dx.doi.org/10.1073/pnas.0711986105 (March 18, Epub 2008 Mar. 10).
Golomb, B. A., 2012. Nature Precedings (bhttps://urldefense.proofpoint.com/v2/url?u=http-3A_hdlhandlenet_10101_npre201268471&d=CwICaQ&c=imBPVzF25OnBgGmVOlcsiEgHo G1i6YHLR0Sj_gZ4adc&r=iHmAiMoon7jduzun9JvyDQ&m=x40ppif6elYWH1YPhTLdAqgaPt Av7×27hsadw81ZGiU&s=D1vsxIho40UmUs60-dvdieCCIpZ8SWvxZm-HdPLv5pY&e=N).
Golomb, B. A., et al., 2014. Neural Comput. 26, 2594-2651.
Grigoryan, H., Lockridge, O., 2009. Toxicol. Appl. Pharmacol. 240 (2), 143-149.
Gupta, R. P., Abou-Donia, M. B., 1995a. Brain Res. 677, 162-166.
Gupta, R. P., Abou-Donia, M. B., 1995b. Neurochem. Res. 20 (9), 1095-1105.
Gupta, R. P., Abdel-Rahman, A., Wilmarth, K. W., Abou-Donia, M. B., 1997. Biochem. Pharmacol. 53, 1799-1806.
Gupta, R. P., Bing, G., Hong, J.-S., Abou-Donia, M. B., 1998. Mol. Cell. Biochem. 181, 29-39.
Gupta, R. P., Abdel-Rahman, A., Jensen, K. F., Abou-Donia, M. B., 2000. Brain Res. 878, 32-47.
Hayakawa, K., Esposito, E., Wang, X., Terasaki, Y., Liu, Y., Xing, C., Ji, X., Lo, E. H., 2016. Nature 535: 551-555. http://dx.doi.org/10.1038/nature18928 (28 July).
Heaton, K. J., Palumbo, C. L., Proctor, S. P., Killiany, R. J., Yurgelun-Todd, D. A., White, R. F., 2007. Neurotoxicology 28, 761-769.
Institute of Medicine, 2012. The National Academic Press, Washington, D.C.
Jacobson, E. E., Meleger, A. L., Bonato, P., et al., 2015. Evid. Based Complement. Alternat. Med. 2015, 813418.
Jauch, E. C., Mayer, S. A., Diringer, M. N., Brun, N. C., Begtrup, K., Steiner, T., Davis, S., Skolnick, B. E., Broderick, J., 2006. J. Emerg. Med. 30 (2) (229-32 230).
Jensen, K. F., Lapadula, D. M., Anderson, J. K., Haykal-Coates, N., Abou-Donia, M. B., 1992. J. Neurosci. Res. 33, 455-460.
Jiang, W., Duysen, E. G., Hansen, H., Shlyakhtenko, L., Schopfer, L., Lockridge, O., 2010. J. Toxicol. Sci. 115, 183-193.
Johnson, G. J., Slater, B. C., Leis, L. A., Rector, T. S., Bach, R. R., 2016. PLoS One 11 (6), e0157855. http://dx.doi.org/10.1371/journal.pone.0157855 (June 28, eCollection 2016).
Kang, H. K., Li, B., Mahan, C. M., Eisen, S. A., Engel, C. C., 2009. J. Occup. Environ. Med. 51 (4), 401-410 (January).
Kaur, P., Radotra, B., Minz, R. W., Gill, K. D., 2007. Neurotoxicology 28, 1208-1219.
Koslik, H. J., et al., 2014. PLoS One 9, e92887.
Laetz, C. A., et al., 2009. Environ. Health Perspect. 117 (3), 348-353.
Lal, H., Forster, M. J., 1988. Neurobiol. Aging 9, 733-742.
Lamproglou, I1., Barbier, L., Diserbo, M., Fauvelle, F., Fauquette, W., Amourette, C., 2009. Behav. Brain Res. 197 (2), 301-310.
Lapadula, D. M., Abou-Donia, M. B., 1992. In: Abou-Donia, M. B. (Ed.), Neurotoxicology Book. CRC Press Boca Raton, pp. 45-59 Chapter 1.
Li, B., Mahan, C. M., Kang, H. K., Eisen, S. A., Engel, C. C., 2011. Am. J. Epidemiol. 174 (7), 761-768 (October 1).
Liliang, Po-C, Cheng-Loong, Liang, LuKuo-Wei, Kang, Wang Hui-Ching, Weng, Ching-Hsieh Li, B., Mahan, C. M., Kang, H. K., Eisen, S. A., Engel, C. C., 2011. Am. J. Epidemiol. 174 (7), 761-768 (October 1).
Maier, S. F., Watkins, L. R., 1998. Psychol. Rev. 105 (1), 83-107 (January, Review).
McMurray, C. T., 2000. Cell Death Differ. 7, 861-865.
Mecocci, P., Parnetti, L., Romano, G., Scarelli, A., Chionne, F., Cecchetti, R., Polidori, M. C., Palumbo, B., Cherubini, A., Senin, U., 1995. J. Neuroimmunol. 57 (1-2), 1651-1670.
Middlemore-Risher, M., Adam, B., Lambert, N. A., Terry Jr., A. V., 2011. J. Pharmacol. Exp. Ther. 339 (2), 341-349.
Milligan, E. D., Watkins, L. R., 2009. Nat. Rev. Neurosci. 10 (1), 23-36 (January).
Nedergaard, M., 2013. Science 340, 1529-1530.
Norgren, N., Rosengren, L., Stigbrand, T., 2003. Brain Res. 987, 25-31.
O'Callaghan, J. P., Sriram, K., 2005 May. Expert Opin. Drug Saf. 4 (3), 433-442.
O'Callaghan, J. P., Kelly, K. A., Locker, A. R., Miller, D. B., Lasley, S. M., 2015. J. Neurochem. 133 (5), 708-721.
Ojo, J. O., Abdullah, L., Evans, J., Reed, J. M., Montague, H., Mullan, M. J., Crawford, F. C., 2014. Neuropathology 34 (2): 109-127. http://dx.doi.org/10.1111/neup.12061 (April, Epub 2013 Sep. 30).
Ozakinci, G., Hallman, W. K., Kipen, H. M., October 2006 Environ. Health Perspect. 114 (10), 1553-1557.
Patton, S. E., O'Callaghan, J. P., Miller, D. B., Abou-Donia, M. B., 1983. J. Neurochem. 41, 897-901.
Patton, S. E., Lapadula, D. M., O'Callaghan, J. P., Miller, D. B., Abou-Donia, M. B., 1985. J. Neurochem. 45, 1567-1577.
Patton, S. E., Lapadula, D. M., Abou-Donia, M. B., 1986. Relationship of Tri-o-cresyl Phosphate-induced Neurotoxicity to Enhancement of In Vitro Phosphorylation of Hen Brain and Spinal Cord Proteins.
Prendergast, M. A., Self, R. L., Smith, K. J., Ghayoumi, L., Mullins, M. M., Butler, T. R., Buccafusco, Gearhart, D. A., Terry Jr., A. V., 2007. Neuroscience 146 (1), 330-339.
Proctor, S. P., et al., 2006. Neurotoxicology 27 (6), 931-939.
Reagan, K. E., Wilmarth, K. R., Friedman, M., Abou-Donia, M. B., 1994. Neurochem. Int. 25, 133-143.
Rempe, D. A., Nedergaard, M., 2010. Neurotherapeutics 7, 335-337.
Research Advisory Committee on Gulf War Veterans Illnesses, (RAC), 2008. Gulf War Illness and the Health of GulfWar Veterans: Scientific Findings and Recommendations.
Research Advisory Committee on Gulf War Veterans Illnesses, (RAC), 2016. Gulf War Illness and the Health of GulfWar Veterans: Scientific Findings and Recommendations.
Schachner, M., Bartsch, U., 2000. Glia 29 (2), 154-165.
Schmahmann, J. D., Smith, E. E., Eichler, F. S., Filley, C. M., 2008. Ann. N. Y. Acad. Sci. 1142:266-309. http://dx.doi.org/10.1196/annals.1444.017 (October, Review).
Schwartz, M., Shechter, R., 2010a. Mol. Psychiatry 15 (4):342-354. http://dx.doi.org/10.1038/mp.2010.31 (April).
Schwartz, M., Shechter, R., 2010b. Nat. Rev. Neurol. 6 (7):405-410. http://dx.doi.org/10.1038/nrneurol.2010.71 (July, Epub 2010 Jun. 8).
Serrano, L., Hernandez, M. A., Avila, J., 1986. J. Biol. Chem. 261, 10332-10339.
Smith, B. N., Wang, J. M., Vogt, D., Vickers, K., King, D. W., King, L. A., 2013. J. Occup. Environ. Med. 55 (1), 104-110 (January).
Soltaninejad, K., Abdollahi, M., 2009 March Med. Sci. Monit. 15 (3), RA75-RA90.
Stalnacke, B. M., Tegner, Y., Sojka, P., 2004. Brain Inj. 18 (9), 899-909.
Stalnacke, B. M., Ohlsson, A., Tegner, Y., Sojka, P., 2006. Br. J. Sports Med. 40 (4), 313-316.
Steele, L., 2000. Am. J. Epidemiol. 152, 992-1002.
Stroick, M., Fatar, M., Ragoschke-Schumm, A., Fassbender, K., Bertsch, T., Hennerici, M. G., 2006. Curr. Med. Chem. 13 (25), 3053-3060 (Review).
Sullivan, K., Krengel, M., Proctor, S. P., Devine, S., Heeren, T., White, R. F., 2003. J. Psychopathol. Behav. Assess. 25, 95-103.
Sullivan, K., Krengel, M., Janulewicz, P., Chamberlain, J., 2013. In: Amara, J., Hendricks, A. (Eds.), Military Medical Care: From Predeployment to Post-separation. Routledge, Abingdon, Va.
Terry Jr., A. V., 2012. Pharmacol. Ther. 134 (3):355-365. http://dx.doi.org/10.1016/j.pharmthera.2012.03.001 (June, Epub 2012 Mar. 20).
Terry Jr., A. V., et al., 2003. J. Pharmacol. Exp. Ther. 305 (1), 375-384.
Terry Jr., A. V., et al., 2012. Neurotoxicol. Teratol. 34 (1), 1-8.
Tuck, M. K., Chan, D. W., Chia, D., et al., 2009. J. Proteome Res. 8, 113-117.
Uda, K1., Goto, K., Ogata, N., Izumi, N., Nagata, S., Matsuno, H., 1998. Neurol. Med. Chir. (Tokyo) 38 (3), 143-152.
Vojdani, A., Thrasher, J. D., 2004. Cellular and humoral immune abnormalities in Gulf War veterans. Environ. Health Perspect. 112, 840-846.
Watkins, L. R., Maier, S. F., 2003. Nat. Rev. Drug Discov. 2 (12), 973-985 (December, Review).
White, R. F., Steele, L., O'Callaghan, J. P., Sullivan, K., Binns, J. H., Golomb, B. A., Bloom, F. E., Bunker, J. A., Crawford, F., Graves, J. C., Hardie, A., Klimas, N., Knox, M., Meggs, W., Melling, J., Philbert, M. A., Grashow, R., 2016. Cortex 74 (January):449-475. http://dx.doi.org/10.1016/j.cortex.2015.08.022 (epub 2015 Sep. 25).
Zakirova, Z., Tweed, M., Crynen, G., Reed, J., Abdullah, L., Nissanka, N., Mullan, M., Mullan, M. J., Mathura, V., Crawford, F., Ait-Ghezala, G., 2015. PLoS One 10 (3), e0119579. http://dx.doi.org/10.1371/journal.pone.0119579 (March 18, eCollection 2015).
Zurek, J., Fedora, M., 2011. J. Trauma (4), 854-859.
Zurek, J., Fedora, M., 2012. Iran J. Med. Sci. 37 (2), 100-104.

### Example 2: Biomarker Signatures for Parkinson's Disease

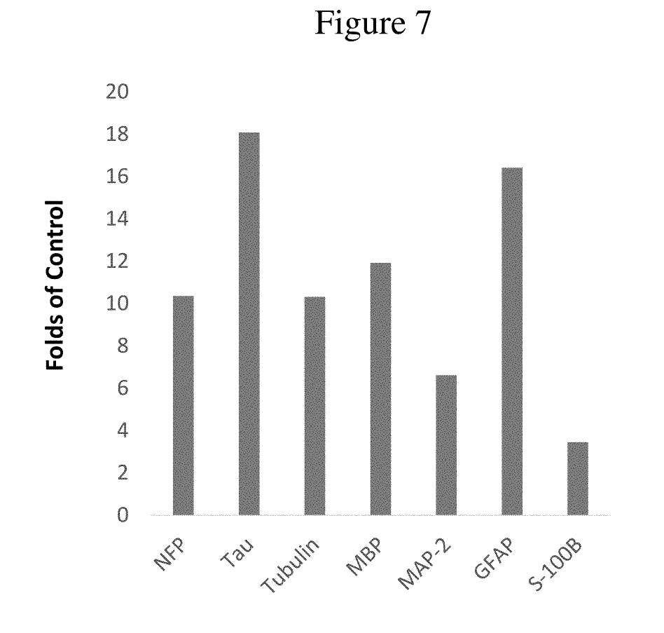

In Parkinson's disease neurons that produce the neurotransmitter dopamine die off in the basal ganglia, an area of the brain that controls body movements. We investigated the relative levels of autoantibodies in the serum of a patient with Parkinson's disease to the neural proteins—NFP, Tau, Tubulin, MBP, MAP-2, GFAP, and S-100B using Western Blot. See FIG. 7. As shown in the Figure autoantibodies specific for each of the proteins were increased in patients with Parkinson's Disease as compared to controls. NFP, Tau, Tubulin, MBP and GFAP were all increased by at least 10 fold and these may be biomarkers for identifying or diagnosing Parkinson's Disease.

### Example 3: Biomarker Signatures for Organophosphate Exposure

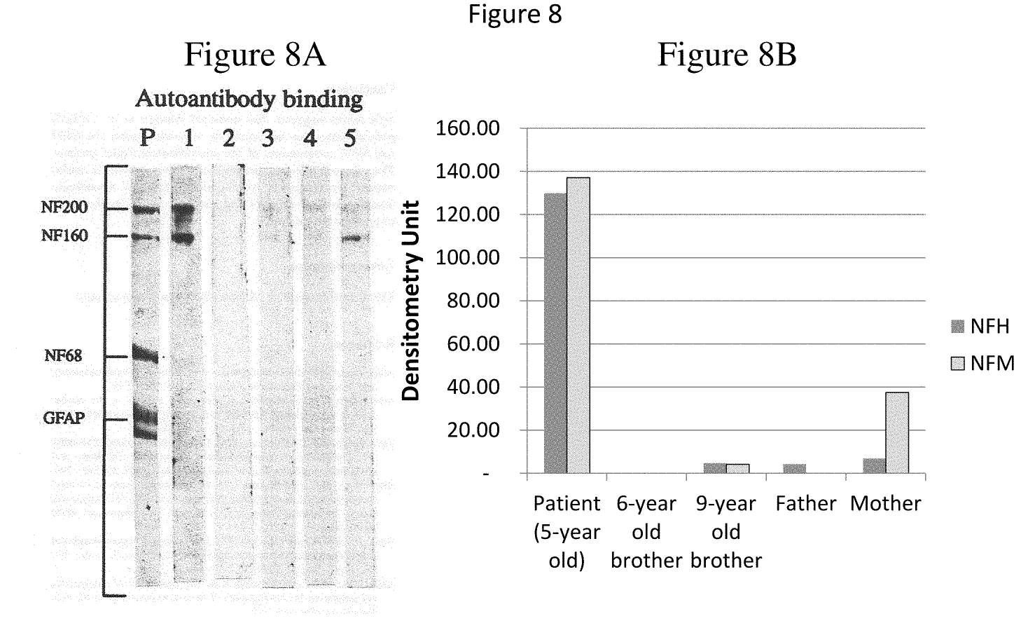

Using Western Blot, we investigated the relative levels of autoantibodies in the serum of a 5-year old patient that had been exposed to the organophosphorus insecticide, chlorpyrifos, to neurofilament proteins—NF200 (NFH), NF160 (NFM), and NF68 (NFL). See FIG. 8. These levels were compared with other members of the patient's immediate family including a 6 year old brother, a 9 year old brother, the patient's father, and the patient's mother.

These results revealed a significant increase in the patient exposed to chlorpyrifos in autoantibodies to NFM and NFH proteins that represent the middle and outer layers of neurofilament triplet proteins. No autoantibodies against the core NFL protein were detected in this patient. These results suggest that neurofilament triplet proteins were partially degraded, and/or NFL protein was completely degraded with no trace. The results of significant increase of neuronal and glial autoantibodies are consistent with axonal degeneration of the nervous system. The results are further in agreement with the action of chlorpyrifos in causing Organophosphate-induced delayed neuropathy (OPIDN), characterized by central/peripheral axonopathy and accompanied by axonal and myelin degeneration. Thus presence of autoantibodies specific for NFH and NFM are indicative of and may be used to diagnose OPIDN.

### Example 4: Biomarker Signatures for Aircraft Fume Exposure

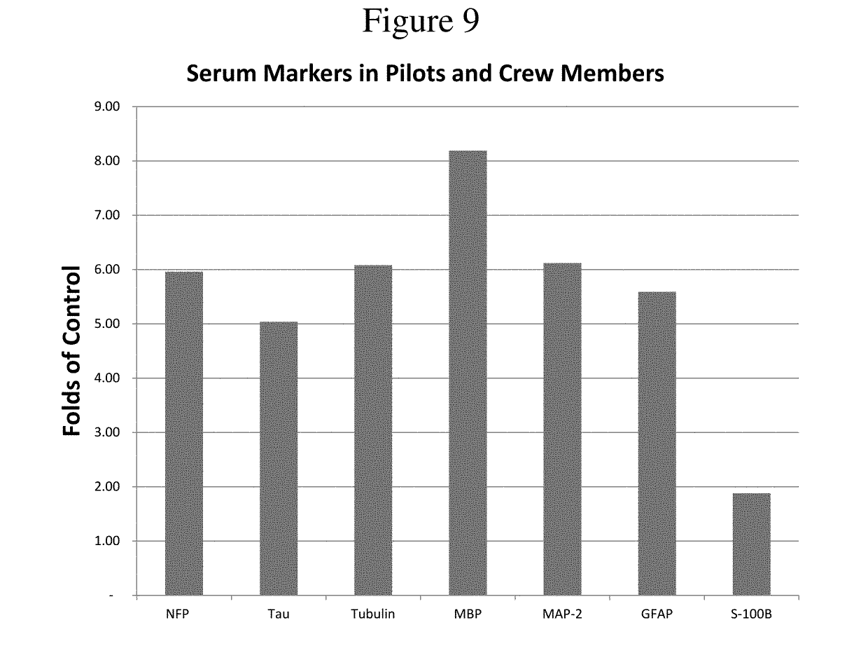

Using Western Blost, we investigated the relative levels of autoantibodies to neural proteins (NFP, Tau, Tubulin, MBP, MAP-2, GFAP, and S-100B) in the serum of a group of 34 pilots and flight attendants that had allegedly been exposed to air emissions (engine oil contaminants, i.e., gaseous, vapor, and particulate constituents of pyrolyzed engine oil) in the unfiltered ventilation air supply that is extracted from either the aircraft engines or auxiliary power unit (APU). See FIG. 9. These levels were compared to a matched group of 12 healthy controls. As shown in the Figure, the levels of autoantibodies to all of the proteins tested except S100B were increased by at least 5 fold as compared to control individuals after prolonged exposure to aircraft fumes.

The results of increased autoantibodies against neuronal and glial proteins are consistent with exposure to organophosphastes, histopathological feature of Ops neurotoxicity, and symptoms resulting from exposure to Ops. This study confirms the allegations that exposure of pilots and flight attendants to fumes in planes might have caused the pilots and cabin crews' illnesses.

### Example 5: Biomarker Signatures for Arsenic-Induced Neural Injury

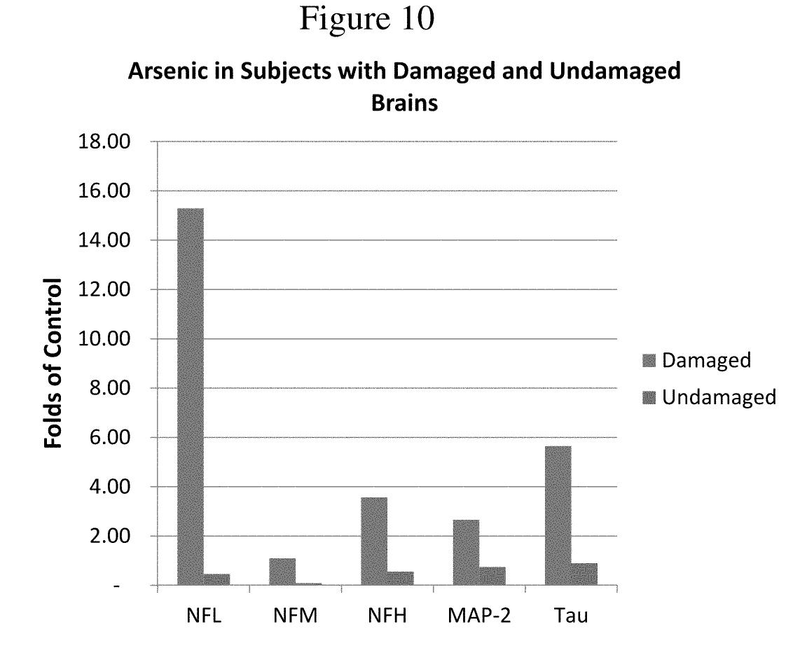

Using Western Blot, we investigated the relative levels of autoantibodies to neural proteins (NFL, NFM, NFH, MAP-2, and Tau) in the serum of a group of 14 subjects from a highly Arsenic-contaminated village of Mianpur in Bangladesh. See FIG. 10. These levels were compared to a matched group of 8 healthy controls. As shown in FIG. 10, the levels of autoantibodies to NFL and Tau were increased by over 5 fold as compared to controls not exposed to arsenic.

The results demonstrated good correlation between levels of serum autoantibodies and arsenic-induced brain injury. The core neurofilament protein NFL exhibited more than 9-fold increase over that of controls. This was followed by significant increase in autoantibodies against Tau, NFH, and MAP-2. Autoantibodies against NFM were not different from controls. Subjects with arsenic poisoning, but no nervous system damage showed no increase in serum neural autoantibodies.

### Example 6: Biomarker Signatures for Stroke

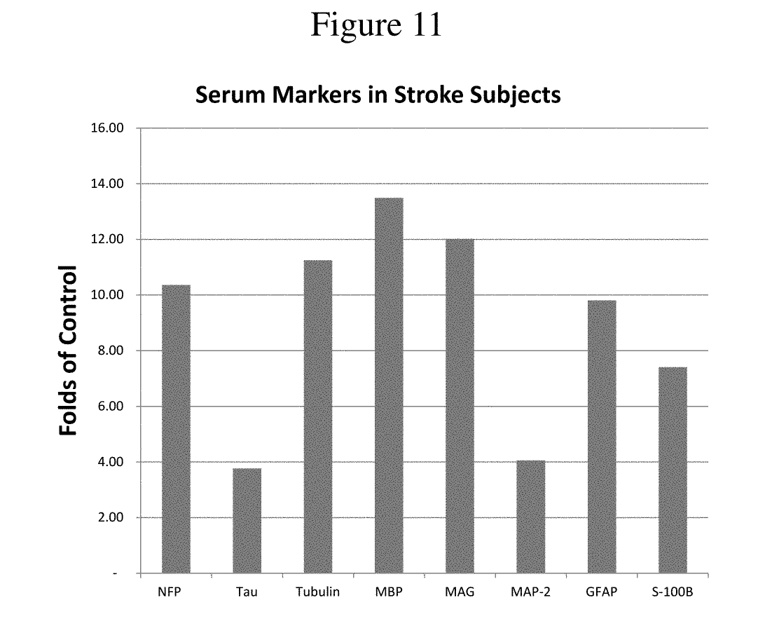

Using Western Blot, we investigated the relative levels of autoantibodies to neural proteins (NFP, Tau, Tubulin, MBP, MAG, MAP-2, GFAP, and S-100B) in the serum of a group of subjects that had a stroke. See FIG. 11. These levels were compared to a matched group of healthy controls. In particular the autoantibody levels directed to NFP, Tubulin, MBP, MAG, and GFAP were all at least 9 fold increase as compared to control subjects.

The results of highly significant increase in neuronal autoantibodies against neuronal proteins indicate brain injury due to stroke.

### Example 7: Biomarker Signatures for Traumatic Brain Injury

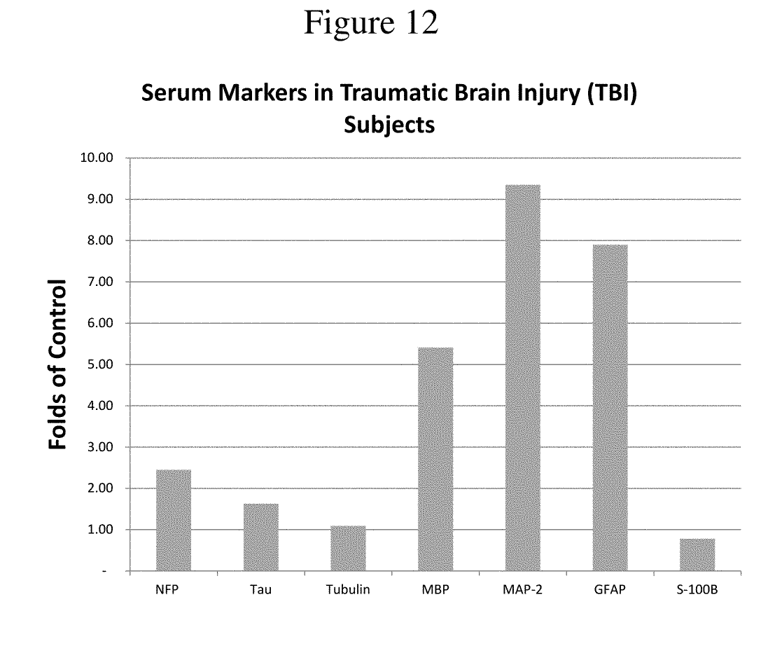

Using Western Blot, we investigated the relative levels of autoantibodies to neural proteins (NFP, Tau, Tubulin, MBP, MAP-2, GFAP, and S-100B) in the serum of a group of 10 subjects with Traumatic Brain Injury (TBI). See FIG. 12. These levels were compared to a matched group of 8 healthy controls.

The results of highly significant increase in neuronal autoantibodies against neuronal proteins indicate brain injury. In particular autoantibodies to MBP, MAP-2 and GFAP were all increased by over 5 fold as compared to controls. The insignificant increase of autoantibodies to S100B suggests that TBI was not a recent injury and that some time has passed allowing S100B to exert its neurotrophic action.

### Example 8: Biomarkers Signatures for Autism

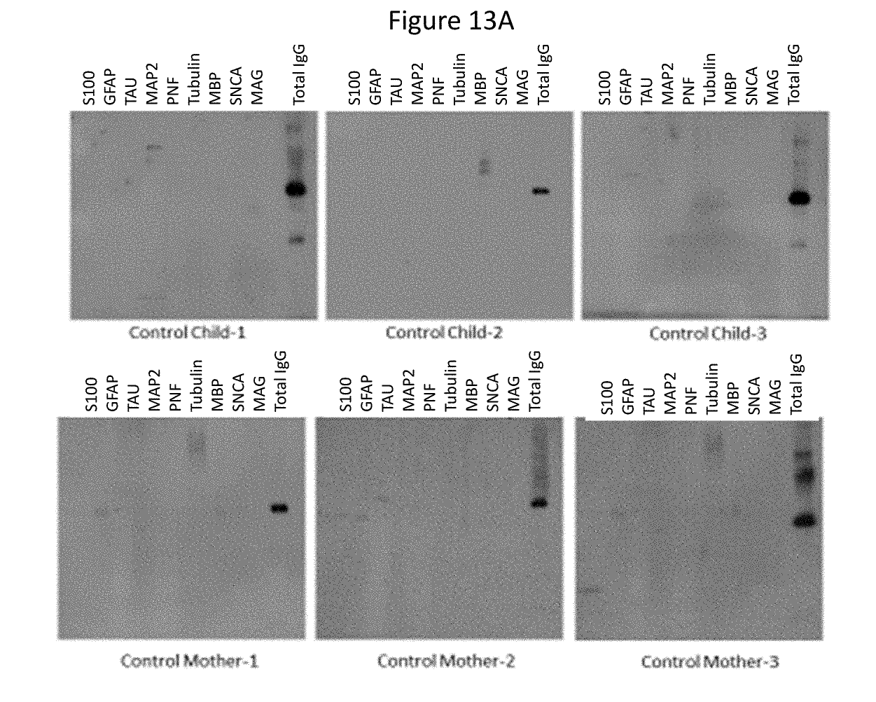

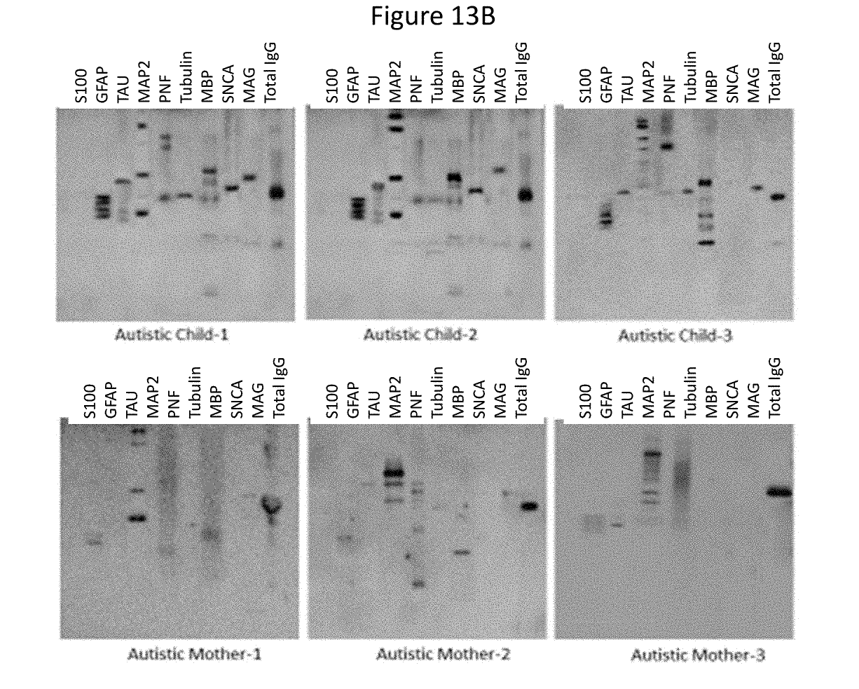

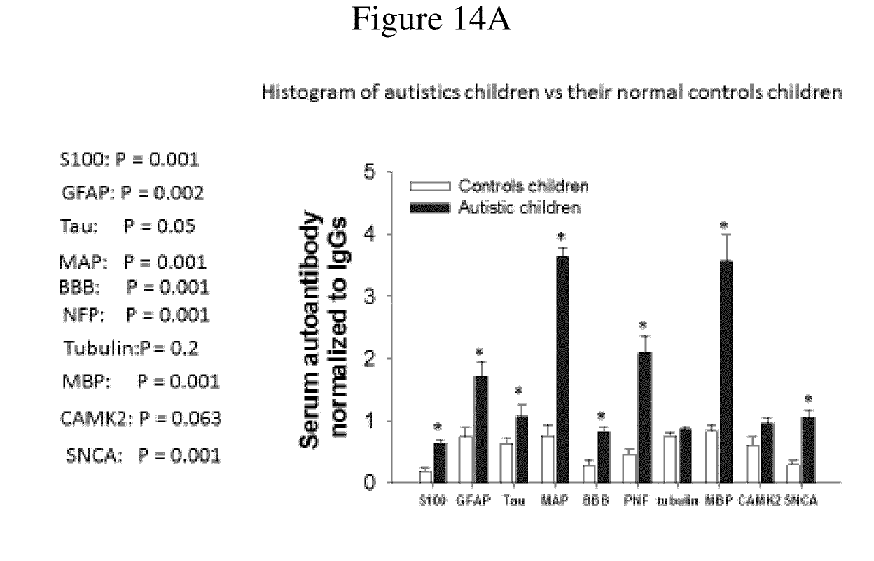

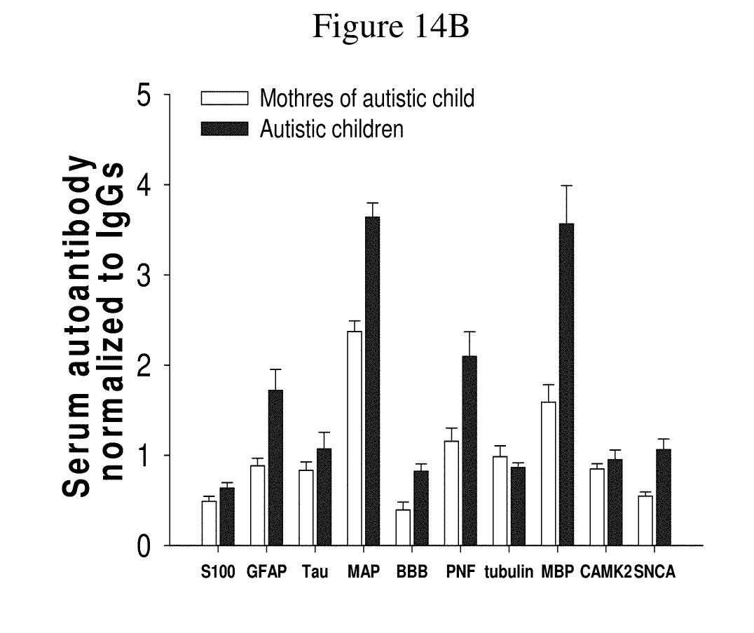

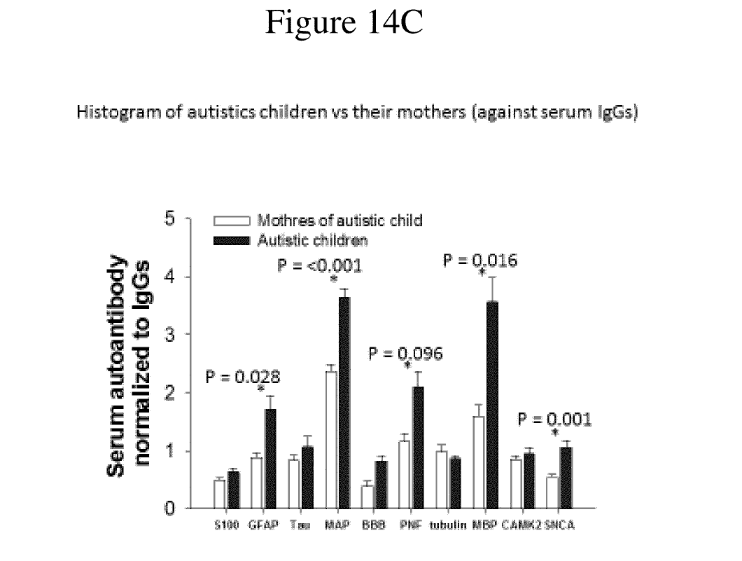

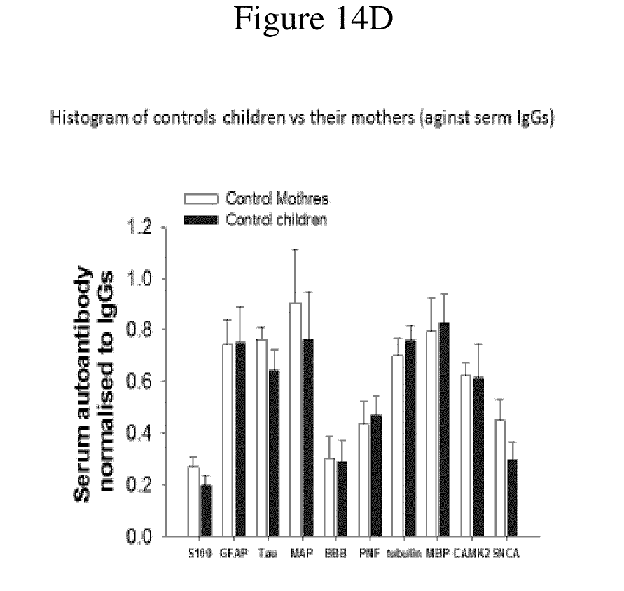

This Example reports the results of assays performed to detect circulating autoantibodies to a panel of 10 proteins associated with the nervous system in sera of 10 children with autism and their mothers and 10 age-matched healthy children and their mothers as controls. The proteins used were chosen to represent the various types of proteins present in nerve cells and affected by neuronal degeneration. In serum samples from all groups, using Western blotting, immunoglobin IgG were measured against the neuronal proteins: neurofilament triplet proteins (NFP or PNF in the figure), tubulin, microtubule associated tau proteins (tau), microtubule associated protein-2 (MAP-2), myelin basic protein (MBP), myelin associated glycoprotein (MAG or BBB in the figure), calcium calmodulin kinase II (CaMKII), and α-synuclein (SNCP), and astrocytic proteins: glial fibrillary acidic protein (GFAP), and S100B protein. See FIGS. 13 and 14.

The results show significantly elevated levels of circulating IgG-class autoantibodies in the children with autism group, compared to controls. Fold increase of autoantibodies against neural proteins for autistic children relative to controls in descending order were: MAP-2 4.78, NFP 4.45, MBP 4.29, MAG 3.83, α-synuclein 3.6, S100 3.2, GFAP 2.29, Tau 1.70, CaMKII 1.55. See FIG. 14A. In contrast, autoantibodies against Tau and tubulin were not statistically significant different from controls. Autistic children's mothers showed less increased levels of autoantibodies compared their autistic children against all neural proteins except for Tau and CaMKII that were not significantly different from controls. See FIG. 14B. We hypothesize that some cases of ASD may be influenced, or even caused, by maternal autoantibodies to neural proteins. Also, this preliminary study suggests that these serum circulating autoantibodies may be used as biomarkers for and/to confirm diagnosis of autism.

1. A method of diagnosing nervous system injury or disease in a subject comprising:
obtaining a sample from the subject;
measuring the level of autoantibody in the sample capable of binding a protein selected from the group consisting of glial fibrillary acidic protein (GFAP), microtubule associated tau protein (Tau), microtubule associated protein-2 (MAP-2), myelin associated glycoprotein (MAG), calcium-calmodulin kinase II (CaM-KII), myelin basic protein (MBP), neurofilament triplet protein (NFP), NF200 (NFH), NF160 (NFM), NF68 (NFL), tubulin, α-synuclein (SNCA), and S100B protein;
comparing the level of the autoantibody in the sample to a reference level of the autoantibody; and
diagnosing the subject with brain injury if the level of the autoantibody is altered as compared to the reference level.

2. The method of claim 1, wherein the subject is a human who served in the military in the Persian Gulf region.

3. The method of claim 1, wherein the level of autoantibody capable of binding at least two of the proteins are measured.

4. The method of claim 3, wherein the ratio of at least two levels of autoantibodies as compared to the reference level allows for a differential diagnosis of nervous system injury resulting from Gulf War Illness from other brain injury, a differential diagnosis of nervous system injury resulting from Traumatic Brain Injury (TBI) from other brain injury, a differential diagnosis of nervous system injury resulting from exposure to an environmental toxin (organophosphate, toxic fumes or arsenic) from other brain injury, a differential diagnosis of Parkinson's disease from other brain injury, a differential diagnosis of stroke from other brain injury, or a differential diagnosis of autism from other brain injury.

5. The method of claim 1, wherein at least two levels of autoantibodies capable of binding at least two of the proteins selected from the group consisting of GFAP, Tau, MAP-2, MAG, CaM-KII, tubulin, and S100B are measured.

6. The method of claim 1, wherein the level of autoantibodies capable of binding GFAP, Tau, MAP-2, MAG, tubulin, CaM-KII, and S100B proteins are measured.

7. The method of claim 1, wherein the level of autoantibodies capable of binding GFAP, tau, tubulin, and CaM-KII proteins are measured.

8. (canceled)

9. The method of claim 1, further comprising administering an immunosuppressant agent or an anti-inflammatory agent to the subject if the subject is diagnosed with nervous system injury or disease.

10. (canceled)

11. The method of claim 1, wherein the sample is serum or plasma.

12. The method of claim 1, wherein the nervous system injury or disease comprises a condition selected from the group consisting of Gulf War Illness, Parkinson's Disease, stroke, autism, Traumatic Brain Injury (TBI), and nervous system damage due to exposure to an organophosphates, exhaust fumes or arsenic.

13. (canceled)

14. (canceled)

15. The method of claim 1, wherein the level of the autoantibody specific for one of the proteins is indicative of the type of nervous system injury or disease.

16. The method of claim 15, wherein autoantibodies specific for at least three of the proteins selected from the group consisting of GFAP, Tau, tubulin, MAP, MBP, NFP, MAG, CAMKII are measured and if the levels are at least 2 fold higher in the subject than the reference levels of autoantibodies then the subject is diagnosed with Gulf War Illness; or wherein autoantibodies specific for at least two of MBP, MAP-2, GFAP or S100B are increased as compared to the reference levels of autoantibodies then the subject is diagnosed with TBI; or wherein autoantibodies specific for at least three of NFP, Tau, tubulin, MBP and GFAP are measured and if the levels of autoantibodies are at least 5 fold higher in the subject than the reference levels, then the subject is diagnosed with Parkinson's Disease; or wherein autoantibodies specific for at least one of NFM and NFH are measured and if the levels of autoantibodies are increased in the subject as compared to the reference levels, then the subject is diagnosed with organophosphate exposure; or wherein autoantibodies specific for at least three of NFP, Tau, tubulin, MBP, MAP-2 and GFAP are measured and if the levels of autoantibodies are increased in the subject as compared to the reference levels, then the subject is diagnosed with exposure to toxic fumes; or wherein autoantibodies specific for at least one of NFL or Tau are measured and if the levels of autoantibodies are increased in the subject as compared to the reference levels, then the subject is diagnosed with exposure to arsenic; or wherein autoantibodies specific for at least three of NFP, tubulin, MBP, MAG and GFAP are measured and if the levels of autoantibodies are increased in the subject as compared to the reference levels, then the subject is diagnosed with stroke; or wherein autoantibodies specific for at least three of NFP, MBP, MAP-2, MAG, α-synuclein, S100B and GFAP are measured and if the levels of autoantibodies are increased in the subject or in the subject's mother as compared to the reference levels, then the subject is diagnosed with autism.

17.-23. (canceled)

24. A kit comprising at least two proteins selected from the group consisting of GFAP, Tau, MAP-2, MAG, CaM-KII, MBP, NFP (NFM, NFH, NFL), tubulin, α-synuclein (SNCA), and S100B protein, further comprising instructions for performing the method of claim 1.

25. (canceled)

26. (canceled)

27. The kit of claim 24, wherein the kit comprises at least two proteins selected from the group consisting of GFAP, Tau, MAP-2, MAG, CaM-KII, and S100B.

28. The kit of claim 24, wherein the kit comprises GFAP, Tau, CaM-KII, and S100B proteins.

29. (canceled)

30. The kit of claim 24, further comprising an anti-human IgG antibody conjugated to a detectable label.

31. A method comprising obtaining a sample from a subject; and determining the level of autoantibodies capable of binding to at least three proteins selected from the group consisting of GFAP, Tau, MAP-2, MAG, CaM-KII, MBP, NFP (NFM, NFH, NFL), tubulin, α-synuclein (SNCA), and S100B protein in the sample.

32. The method of claim 31, wherein the level of autoantibodies capable of binding to at least five of the proteins are determined in the sample.

33. The method of claim 31, wherein the presence of autoantibodies is indicative of nervous system injury or disease.

34. (canceled)

35. The method of claim 31, further comprising treating the subject determined to have autoantibodies capable of binding at least three of the proteins with an immunosuppressive or analgesic agent.
